(19) United States
(12) Patent Application Publication
(10) Pub. No.: US 2021/0355547 A1
(43) Pub. Date: Nov. 18, 2021

(54) NON-INVASIVE URINARY BIOMARKERS FOR THE DETECTION OF UROTHELIAL CARCINOMA OF THE BLADDER

(71) Applicant: THE JOHNS HOPKINS UNIVERSITY, Baltimore, MD (US)

(72) Inventors: Mohammad O. Hoque, Towson, MD (US); Akira Oki, Baltimore, MD (US)

(21) Appl. No.: 17/286,934

(22) PCT Filed: Oct. 18, 2019
(86) PCT No.: PCT/US2019/056873

§ 371 Date: Apr. 20, 2021

Related U.S. Application Data
(60) Provisional application No. 62/748,403, filed on Oct. 20, 2018.

(51) Int. Cl. C12Q 1/6886; C12Q 1/686

(52) U.S. Cl. CPC C12Q 1/6886; C12Q 2600/158; C12Q 1/686

(57) ABSTRACT

The present invention relates to the field of cancer. More specifically, the present invention provides compositions and methods for detecting urothelial carcinomas. In one embodiment, a method comprises the step of detecting CD24, CD49f and NANOG in a urine sample obtained from a patient suspected of having urothelial carcinoma. In a specific embodiment, the detection step comprises polymerase chain reaction (PCR). In certain embodiment, the PCR step utilizes at least one primer comprising SEQ ID NOS: 1, 2, 23, 24, 13 and 14. In another embodiment, the method further comprises treating the patient with one or more of surgery, radiation therapy, chemotherapy and immunotherapy.

### CROSS-REFERENCE TO RELATED APPLICATIONS

This application claims the benefit of U.S. Provisional Application No. 62/748,403, filed Oct. 20, 2018, which is incorporated herein by reference in its entirety.

### FIELD OF THE INVENTION

The present invention relates to the field of cancer. More specifically, the present invention provides compositions and methods for detecting and treating urothelial carcinomas.

### INCORPORATION-BY-REFERENCE OF MATERIAL SUBMITTED ELECTRONICALLY

This application contains a sequence listing. It has been submitted electronically via EFS-Web as an ASCII text file entitled “P15198-02_ST25.txt.” The sequence listing is 6,986 bytes in size, and was created on Oct. 17, 2019. It is hereby incorporated by reference in its entirety.

### BACKGROUND OF THE INVENTION

Urothelial carcinoma of the bladder (UCB) is the most common malignancy of the urinary tract, with an estimated 79,030 new cases and 16,870 deaths from the disease per year in the United States¹. The five-year relative survival rate is >90% when detected as a non-muscle-invasive bladder cancer (NMIBC), while it drops to <50% for muscle-invasive disease². Diagnosis at early stage of the initial and recurrent disease is crucial for favorable outcomes. However, the estimated recurrence rate of NMIBC is 60-70% and 10%-30% of these patients will progress to muscle-invasive bladder cancer (MIBC) despite curative intensive therapy^{3,4}. This high recurrence rate requires patients to undergo frequent and life-long monitoring. Although the non-invasive, highly-specific urine cytology assay is commonly used for the surveillance of UCB patients, its sensitivity is relatively low, specifically for low-grade tumors⁵⁻⁸. Clinically robust sensitive and specific urinary biomarkers are needed to supplement the urine cytology test.

### SUMMARY OF THE INVENTION

Tumors are hierarchically organized by a rare population of cancer stem cells (CSCs) that contributes to cancer initiation, progression, and treatment failure^{9,10}. A better understanding of the molecular mechanisms underlying urothelial CSC regulation and the identification of key molecules associated with CSC generation and maintenance are pivotal for the determination of biology-based accurate biomarkers for early cancer detection, monitoring following transurethral resection of bladder tumor (TURBT), and molecular-targeting therapy. We recently demonstrated that CD24 is a crucial CSC marker that is overexpressed in urothelial CSCs¹¹. It was also reported previously that CD24 acts as a hub of tumorigenesis and metastatic progression and associated with a poor outcome in UCB¹²⁻¹⁵. Moreover, CD24-deficiency reduced urothelial tumorigenesis and metastasis in a mouse model¹⁶ and treatment with an anti-CD24 monoclonal antibody resulted in a decreased metastatic tumor burden¹⁵. Thus, CD24 has been implicated in tumor initiation and progression as an oncogene, and it is a potential therapeutic target for UCB. However, the oncogenic role of CD24 in UCB is incompletely understood. Although CD24 has been characterized as a major determinant of stemness in other cancer types, including liver ¹⁷ and colorectal carcinoma¹⁸, it is still unclear whether CD24 functionally contributes to urothelial CSC-like traits. Furthermore, although meta-analysis indicated that CD24 is an important marker of malignancy, including for UCB¹², its clinical utility as a biomarker for cancer detection has not been tested yet.

We recently demonstrated that chronic arsenic exposure endows urothelial cells with malignant stemness properties, including increased expression of several CSC-related molecules such as SOX2, CD24, and NANOG¹⁹. Furthermore, we observed incremental expression of SOX2 in urine samples from carcinogen (arsenic) exposed non-cancer subjects and UCB subjects compared with urine samples from non-exposed control subjects. Given these findings and the central role of CSCs at the top of the cellular hierarchy in tumor initiation, we hypothesized that urothelial CSC-related molecules may serve as urinary biomarkers for discriminating between subjects with and without UCB. In this study, we performed a quantitative expression assessment of 15 CSC-related molecules in urine samples from 24 non-cancer control and 24 UCB subjects to construct a candidate panel of urinary biomarker for UCB detection. After determining the analytical and clinical sensitivity of a panel of 3 genes (CD24, CD49f, and NANOG) in a set of primary tumor with the matched urine, we evaluated the clinical utility of this panel of three CSC-related molecules in an independent cohort of urine samples from 60 UCB and 183 control subjects. Furthermore, we evaluated the functional contribution of CD24 to urothelial cancer stem-like traits using in vitro and in vivo approaches.

Accordingly, in one aspect, the present invention provides methods for detecting urothelial biomarkers. In one embodiment, a method comprises the step of detecting CD24, CD49f and NANOG in a urine sample obtained from a patient suspected of having urothelial carcinoma. In particular embodiments, the detection step comprises polymerase chain reaction (PCR). In a specific embodiment, the PCR utilizes at least one primer comprising SEQ ID NOS: 1, 2, 23, 24, 13 and 14. In certain embodiments, the PCR comprises quantitative real time PCR (RT-PCR). In a specific embodiment, the RT-PCR parameters comprise 50° C. for 2 min., 95° C. for 10 min., followed by 40 cycles at 95° C. for 15 sec. and 58° C. for 1 min. In another embodiment, the method further comprises at least one of ALDH1A1, Bmi1, OCT4, CK14, CD133, CD44, CD90, HMGA2, YAP1, and ABCG2. In certain embodiments, the detecting step comprises PCR and utilizes at least one primer comprising SEQ ID NOS:5-10, 17-32.

The method can further comprise treating the patient with one or more of surgery, radiation therapy, chemotherapy and immunotherapy. In particular embodiments, surgery comprises one or more of transurethral resection, radical cystectomy, partial cystectomy, lymph node dissection and urinary diversion. In other embodiments, chemotherapy comprises cisplatin-based chemotherapy or carboplatin-based chemotherapy. In a specific embodiment, immunotherapy comprises a checkpoint inhibitor. In more specific embodiments, the checkpoint inhibitor comprises one or more of ipilimumab, nivolumab, pembrolizumab, atezolizumab, avelumab and durvalumab.

In another aspect, the present invention provides methods for detecting urothelial carcinoma in a patient. In one embodiment, a method comprises the step of detecting higher expression of CD24, CD49f and NANOG in a urine sample obtained from the patient relative to a control. In a specific embodiment, the detection step comprises polymerase chain reaction (PCR). In a more specific embodiment, the PCR utilizes at least one primer comprising SEQ ID NOS: 1, 2, 23, 24, 13 and 14. The PCR can comprise RT-PCR. In specific embodiments, the RT-PCR parameters comprise 50° C. for 2 min., 95° C. for 10 min., followed by 40 cycles at 95° C. for 15 sec. and 58° C. for 1 min. In other embodiments, the method further comprises detecting at least one of ALDH1A1, Bmi1, OCT4, CK14, CD133, CD44, CD90, HMGA2, YAP1, and ABCG2. In specific embodiments, the detecting step comprises PCR and utilizes at least one primer comprising SEQ ID NOS:5-10, 17-32.

In another aspect, the present invention provides methods for treating urothelial carcinoma in a patient. In one embodiment, the method comprises the steps of (a) detecting higher expression of CD24, CD49f and NANOG in a biological sample obtained from the patient relative to a control; and (b) treating the patient with one or more of surgery, radiation therapy, chemotherapy and immunotherapy. In particular embodiments, surgery comprises one or more of transurethral resection, radical cystectomy, partial cystectomy, lymph node dissection and urinary diversion. In another embodiment, chemotherapy comprises cisplatin-based chemotherapy or carboplatin-based chemotherapy. In a specific embodiment, immunotherapy comprises a checkpoint inhibitor. In more specific embodiments, the checkpoint inhibitor comprises one or more of ipilimumab, nivolumab, pembrolizumab, atezolizumab, avelumab and durvalumab.

In certain embodiments, the biological sample comprises urine, blood, plasma, serum, and saliva. In a specific embodiment, wherein the detection step comprises polymerase chain reaction (PCR). In a more specific embodiment, the PCR utilizes at least one primer comprising SEQ ID NOS: 1, 2, 23, 24, 13 and 14. In particular embodiments, the PCR comprises RT-PCR. In specific embodiments, the RT-PCR parameters comprise 50° C. for 2 min., 95° C. for 10 min., followed by 40 cycles at 95° C. for 15 sec. and 58° C. for 1 min. In other embodiments, the method further comprises detecting at least one of ALDH1A1, Bmi1, OCT4, CK14, CD133, CD44, CD90, HMGA2, YAP1, and ABCG2. In specific embodiments, the detecting step comprises PCR and utilizes at least one primer comprising SEQ ID NOS:5-10, 17-32.

### BRIEF DESCRIPTION OF THE FIGURES

FIG. 1A-1D. The sphere-forming and invasive abilities attenuated by CD24 knockdown in BFTC 905, BFTC 909, and 5637 cell lines. (FIG. 1A) Western blotting analysis of CD24 in isogeneic parental and spheroid cells. Upper, western blotting images; Lower, expression levels of CD24 were quantified by myImageAnalysis™ Software and normalized to their respective β-actin. (FIG. 1B) Western blotting analysis of CD24 in stable CD24 knockdown (CD24-sh) and control (CD24-Ctrl) cells. Upper, western blotting images; Lower, expression levels of CD24 were quantified relative to β-actin. (FIG. 1C) Sphere formation and self-renewal assays through the second (P2) passage from the first passage (P1) in stable CD24-sh cells compared with control CD24-Ctrl cells. Upper, representative images of sphere formation (scale bars, 200 μm); Lower, number of the spheres over 100 μm. Data are from three independent experiments. (FIG. 1D) Invasion assay of spheroid CD24-sh cells. Upper: representative images (scale bars, 100 μm). Lower: the relative number of invaded spheroid CD24-sh cells compared with spheroid CD24-Ctrl cells. Each error bar indicates mean±SEM. *, P<0.05; **, P<0.01 (Wilcoxon-Mann-Whitney test).

FIG. 2A-2E. The chemoresistant and tumorigenic abilities attenuated by CD24 knockdown in BFTC 905, BFTC 909, and 5637 cell lines. (FIG. 2A) Cell viability after 5 μM cisplatin (CDDP) treatment for 72 h in the spheroid CD24-sh cells, as measured by MTT assay. Cell viability was expressed as the ratio of absorbance values of the spheroid CD24-sh cells related to the spheroid CD24-Ctrl cells considered as 1.0. Data are from three independent experiments. (FIG. 2B) An apoptosis assay of spheroid CD24-sh cells treated with 5 μM CDDP for 72 h. Upper, representative images of early apoptosis (bottom right quadrant) and late apoptosis (top right quadrant); Lower, percentage of apoptotic cells. (FIG. 2C) Western blotting analysis of ABCG2, YAP1, and CD133 in stable CD24-sh and CD24-Ctrl cells. (FIG. 2D) Limiting-dilution xenograft assays in stable spheroid CD24-sh BFTC 909 (upper) and BFTC 905 (lower) cells. Tumor growth was measured after subcutaneous injection of serially diluted spheroid cells (1×10⁴, 1×10³, or 1×10² cells per flank) into both flanks of NSG mice (five mice per group). SP, Spheroid. (FIG. 2E) Tumor initiation frequency after the xenotransplantation of spheroid CD24-sh BFTC 909 (upper) and BFTC 905 (lower) cells. Tumor-initiating capacity is shown as the numbers of tumors/the number of injections after 70 days from subcutaneous injection of serially diluted spheroid CD24-sh cells (1×10⁴, 1×10³, or 1×10² cells per flank) into both flanks of NSG mice (five mice per group). Each error bar indicates mean±SEM. *, P<0.05; **, P<0.01 (Wilcoxon-Mann-Whitney test).

FIG. 3A-3E. Enriched cancer stem-like traits in CD24-expressing cells. (FIG. 3A) Flow cytometry analysis after isolation of each subpopulation (high- and low-CD24-expressing cells) from PDX CTG1388 and CTG1061 tumors according to the endogenous status of CD24 expression using the magnetic-activated cell sorting. (FIG. 3B) Sphere formation assay after isolation of high- and low-CD24-expression cells. Upper, representative images of sphere formation (scale bars, 200 μm); Lower, number of the spheres over 100 μm. Data are from three independent experiments. (FIG. 3C) Cell viability after 5 μM cisplatin (CDDP) treatment for 72 h in high- and low-CD24-expressing cells, as measured by MTT assay. Cell viability was expressed as the ratio of absorbance values of high-CD24-expressing cells treated with CDDP related to low-CD24-expressing cells considered as 1.0. Data are from three independent experiments. (FIG. 3D) Association of CD24 with ABCG2, CD133, and YAP1 expression in high-CD24-expressing cells compared with low-CD24-expressing cells. The relative mRNA expression levels of CD24, ABCG2, CD133, and YAP1 in high-CD24-expressing cells were calculated considering the expression values equal to 1.0 in low-CD24-expressing cells, as measured by qRT-PCR. (FIG. 3E) The in vivo tumorigenicity of xenotransplantation of high- and low CD24-expressing CTG1388 (upper) and CTG1061 (lower) cells into both flanks of NSG mice (five mice per group). Left, tumor growth curve after subcutaneous injection of the cells (1×10⁴ cells per flank); Right, representative images of tumors. Each error bar indicates mean±SEM. *, P<0.05 (Wilcoxon-Mann-Whitney test).

FIG. 4A-4E. The cancer detection accuracy of a combination panel of three CSC-related molecules (CD24, CD49f, and NANOG) in urine samples. (FIG. 4A) Box plots of the relative expression levels of CD24 mRNA in 30 primary tumor and the matched adjacent normal tissues. Scatter plots show the distribution of individual expression value of CD24 determined by qRT-PCR. The expression levels of tumor and the matched adjacent tissues were connected with a line. (FIG. 4B) Box plots of the expression levels of CD24 mRNA in19 UCB samples and the matched adjacent, histologically normal samples in the TCGA cohort. The expression values (RSEM log₂) of tumors and the matched adjacent tissues were connected with a line. (FIG. 4C) Box plots of the relative expression levels of CD24 mRNA in urine samples from 24 UCB and 24 control subjects. (FIG. 4D) Sensitivity and specificity of the combination panel for cancer detection in urine samples of the training cohort and independent validation cohort. The schematic representation shows true positives, false negatives, true negatives, and false positive detected by the combination panel of three molecules (CD24, CD49f, and NANOG). (FIG. 4E) Analytical sensitivity (AS) of CD24, CD49f, and NANOG in 17 primary UCB and the matched urine samples. Analytical sensitivity is defined as “The fraction of cases in which overexpression of a marker was found in urine RNA for case patients who had confirmed overexpression of the same marker in the primary tumor RNA”. Cells in color represent the positive expression, defined by optimal cutoff value determined by ROC curve, for each molecule in primary tumor (P) and the matched urine (U) samples. Each data indicates mean±SEM. The paired t-test (A and B) were performed.

FIG. 5. CSC-related molecules modulated by CD24 knockdown in BFTC 905, BFTC 909, and 5637 cell lines. The relative mRNA expression levels of 15 potential CSC-related molecules in CD24-sh cells were calculated considering the expression values equal to 1.0 in CD24-Ctrl cells, as measured by qRT-PCR and normalized by β-actin. The expression levels of ABCG2, CD133, and YAP1 were consistently downregulated due to knockdown of CD24. Data are from three independent experiments. Each error bar indicates mean±SEM.

FIG. 6. The relative expression levels of CD24, CD49f, and NANOG in primary tumor tissues according to their expression status in the matched urine samples. Each data indicates mean±SEM. Wilcoxon-Mann-Whitney test were performed.

FIG. 7. Box plots of the relative expression levels of CD24, CD49f, and NANOG in urine samples from UCB (n=60) and control (n=183) in an independent validation cohort. Data indicates mean±SEM. Wilcoxon-Mann-Whitney test was performed.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

CD24 is a cornerstone of tumor progression in urothelial carcinoma of the bladder (UCB). However, its contribution to cancer stem cell (CSC)-like traits and the clinical utility of CD24 as a urinary biomarker for cancer detection have not been determined. As described herein, the functional relevance of CD24 was evaluated using in vitro and in vivo approaches. The clinical utility of CSC-related molecules was assessed in urine samples by quantitative RT-PCR.

The knockdown of CD24 attenuated cancer stemness properties. The high-CD24-expressing cells, isolated from patient-derived UCB xenograft tumors, exhibited their enhanced stemness properties. CD24 was overexpressed in primary tumors and in urine of UCB subjects. By assessment of 15 candidate CSC-related molecules in urine samples of a training cohort, a panel of three molecules (CD24, CD49f, and NANOG) was selected. The combination of these three molecules yielded a sensitivity and specificity of 81.7% and 74.3% respectively in an independent cohort. A combined set of 84 cases and 207 controls provided a sensitivity and specificity of 82% and 76% respectively.

Thus, CD24 plays a crucial role in maintaining the urothelial cancer stem-like traits, and a panel of CSC-related molecules has potential as a urinary biomarker for non-invasive UCB detection.

Accordingly, the present invention is based, at least in part, on the discovery that combinations of certain urothelial carcinoma markers can be detected in urine. These markers can be detected and used to detect urothelial carcinoma in the bladder (UCB), as well as to treat patients having UCB. Although urine is the preferred embodiment, the markers described herein can be detected in other biological samples including, but not limited to, blood, plasma, serum, saliva and the like.

In one embodiment, a method comprises the step of detecting CD24, CD49f and NANOG in a urine sample obtained from a patient suspected of having urothelial carcinoma. In a specific embodiment, the detection step comprises polymerase chain reaction (PCR). In certain embodiment, the PCR step utilizes at least one primer comprising SEQ ID NOS: 1, 2, 23, 24, 13 and 14. In another embodiment, the method further comprises treating the patient with one or more of surgery, radiation therapy, chemotherapy and immunotherapy.

In particular embodiments, the PCR assay is quantitative-RT-PCR. In certain embodiments, the PCR parameters comprise 50° C. for 2 min, 95° C. for 10 min, followed by 40 cycles at 95° C. for 15 sec and 58° C. for 1 min.

The panel of markers comprising CD24, CD49f and NANOG is but one example of a panel of markers that can be used to detect, risk stratify, treat, and/or monitor treatment of UCB. The primers listed in Table 3 can be used to detect the markers in urine samples obtained from patients. The methods can use at least 1, at least 2, at least 3, at least 4 and so on of the primers listed in Table 3 for markers that include NANOG, CD24, CD49f, LGR5, Np63 and SOX2.

Thus, in certain embodiments, the panel comprises 6 markers. In a specific embodiment, the panel comprises NANOG, CD24, CD49f, LGR5, Np63 and SOX2. The method can comprise detection of the panel using at least one primer comprising SEQ ID NOS:13, 14, 1, 2, 23, 24, 11, 12, 15, 16, 3 and 4.

In other embodiments, the panel comprises 5 markers. In a specific embodiment, the panel comprises NANOG, CD24, CD49f, LGR5 and Np63. The method can comprise detection of the panel using at least one primer comprising SEQ ID NOS:13, 14, 1, 2, 23, 24, 11, 12, 15, and 16. In another embodiment, the panel comprises NANOG, CD24, CD49f, LGR5 and SOX2. The method can comprise detection of the panel using at least one primer comprising SEQ ID NOS:13, 14, 1, 2, 23, 24, 11, 12, 3 and 4.

In particular embodiments, the panel comprises 4 markers. In one embodiment, the panel comprises NANOG, CD24, CD49f and LGR5. The method can comprise detection of the panel using at least one primer comprising SEQ ID NOS:13, 14, 1, 2, 23, 24, 11 and 12. In another embodiment, the panel comprises NANOG, CD24, CD49f and Np63. The method can comprise detection of the panel using at least one primer comprising SEQ ID NOS:13, 14, 1, 2, 23, 24, 15 and 16. In an alternative embodiment, the panel comprises NANOG, CD24, CD49f and SOX2. The method can comprise detection of the panel using at least one primer comprising SEQ ID NOS:13, 14, 1, 2, 23, 24, 3 and 4. In a further embodiment, the panel comprises CD24, CD49f LGR5 and SOX2. The method can comprise detection of the panel using at least one primer comprising SEQ ID NOS:1, 2, 23, 24, 11, 12, 3 and 4. In yet another embodiment, the panel comprises NANOG, CD24, LGR5 and SOX2. The method can comprise detection of the panel using at least one primer comprising SEQ ID NOS:13, 14, 1, 2, 11, 12, 3 and 4. In yet a further embodiment, the panel comprises NANOG, CD24, LGR5 and Np63. The method can comprise detection of the panel using at least one primer comprising SEQ ID NOS:13, 14, 1, 2, 11, 12, 15 and 16. In another embodiment, the panel comprises NANOG, CD24, LGR5 and SOX2. The method can comprise detection of the panel using at least one primer comprising SEQ ID NOS:13, 14, 1, 2, 11, 12, 3 and 4.

In other embodiments, the panel comprises 3 markers. In one embodiment, the panel comprises NANOG, CD24 and CD49f. The method can comprise detection of the panel using at least one primer comprising SEQ ID NOS:13, 14, 1, 2, 23 and 24. In another embodiment, the panel comprises NANOG, CD24 and LGR5. The method can comprise detection of the panel using at least one primer comprising SEQ ID NOS:13, 14, 1, 2, 11 and 12. In an alternative embodiment, the panel comprises NANOG, CD24 and SOX2. The method can comprise detection of the panel using at least one primer comprising SEQ ID NOS:13, 14, 1, 2, 3 and 4. In a further embodiment, the panel comprises NANOG, CD24 and Np63. The method can comprise detection of the panel using at least one primer comprising SEQ ID NOS:13, 14, 1, 2, 15 and 16. In yet another embodiment, the panel comprises NANOG, CD49f and Np63. The method can comprise detection of the panel using at least one primer comprising SEQ ID NOS:13, 14, 23, 24, 15 and 16. In yet a further embodiment, the panel comprises CD24, CD49f and LGR5. The method can comprise detection of the panel using at least one primer comprising SEQ ID NOS:1, 2, 23, 24, 3 and 4. In another embodiment, the panel comprises CD24, CD49f and SOX2. The method can comprise detection of the panel using at least one primer comprising SEQ ID NOS: 1, 2, 23, 24, 3 and 4.

In one aspect, the methods of the present invention can include a treatment step. In certain embodiments, treatment can comprise surgery including, but not limited to, one of transurethral resection (including, for example, with fulguration), radical cystectomy, partial cystectomy, lymph node dissection, and urinary diversion. Treatment can also include radiation therapy (external or internal), as well as chemotherapy. In certain embodiments, treatment comprises photodynamic therapy. In a specific embodiment, treatment comprises transurethral reception with or without intravesical therapy.

In particular embodiments, treatment comprises immunotherapy. Examples of immunotherapy can include, but are not limited to, checkpoint inhibitors such as PD-L1 inhibitors and PD-1 inhibitors. Examples of PD-L1 inhibitors include atezolizumab, durvalumab and avelumab. Examples of PD-1 inhibitors include pembrolizumab and nivolumab. Another form of immunotherapy that can be used as a treatment step includes bacillus Calmette-Guerin (BCG). Other treatment therapies include, but are not limited to, rogaratinib, lapatinib and erlotinib.

In other embodiments, treatment comprises anti-angiogenesis drugs include vascular endothelial growth factor inhibitors including, but not limited to, bevacizumab, sunitinib, cabozantinib and pazopanib. Angiogenesis inhibitors also include sorafenib. In certain embodiments, treatments comprise an anti-angiogenesis drug combined with chemotherapy. In particular embodiments, treatment comprises a combination of bevacizumab with first-line gemcitabine and ramucirumab with second-line docetaxel. In other embodiments, treatment comprises cabozantinib in combination with nivolumab or nivolumab plus ipilimumab.

Treatment steps can also include gemcitabine hydrochloride and cisplatin, enfortumab, sapanisertib, pemigatinib, ipilimumab, ASG-22ME (enfortumab vedotin), as well as combinations of all the foregoing treatments. For example, a treatment step can include CD122-biased cytokine (NKTR-214) in combination with anti-PD-1 antibody (e.g., nivolumab) or in combination with nivolumab and anti-CTLA4 antibody (ipilimumab).

In further embodiments, the treatments comprises one or more chemotherapeutic agents. Anti-cancer drugs that may be used in the various embodiments of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; erlotinib; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gefitinib; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-nl; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; navelbine; nivolumab; nocodazole; nogalamycin; ormaplatin; oxisuran; paclitaxel; pegaspargase; peliomycin; pemetrexed; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, improsulfan, benzodepa, carboquone, triethylenemelamine, triethylenephosphoramide, triethylenethiophosphoramide, trimethylolomelamine, chlornaphazine, novembichin, phenesterine, trofosfamide, estermustine, chlorozotocin, gemzar, nimustine, ranimustine, dacarbazine, mannomustine, mitobronitol, aclacinomycins, actinomycin F(1), azaserine, bleomycin, carubicin, carzinophilin, chromomycin, daunorubicin, daunomycin, 6-diazo-5-oxo-1-norleucine, doxorubicin, olivomycin, plicamycin, porfiromycin, puromycin, tubercidin, zorubicin, denopterin, pteropterin, 6-mercaptopurine, ancitabine, 6-azauridine, carmofur, cytarabine, dideoxyuridine, enocitabine, pulmozyme, aceglatone, aldophosphamide glycoside, bestrabucil, defofamide, demecolcine, elfornithine, elliptinium acetate, etoglucid, flutamide, hydroxyurea, lentinan, phenamet, podophyllinic acid, 2-ethylhydrazide, razoxane, spirogermanium, tamoxifen, taxotere, tenuazonic acid, triaziquone, 2,2′,2″-trichlorotriethylamine, urethan, vinblastine, vincristine, vindesine and related agents. 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer, Preferred additional anti-cancer drugs are 5-fluorouracil and leucovorin, Additional cancer therapeutics include monoclonal antibodies such as rituximab, trastuzumab and cetuximab.

In a specific embodiment, the treatment comprises atezolizumab, gemcitabine and carboplatin/cisplatin. In another embodiment, the treatment comprises durvalumab or durvalumab and tremelimumab. In an alternative embodiment, treatment comprises pembrolizumab and chemotherapy. In a further embodiment, treatment comprises nivolumab and ipilimumab. In another embodiment, treatment comprises gemcitabine hydrochloride, cisplatin and bevacizumab. In yet another embodiment, treatment comprises epacadostat and pembrolizumab.

### Materials and Methods

Cell lines and tissue samples. UCB cell line 5637 was obtained from the American Type Culture Collection (ATCC; Manassas, Va., USA). BFTC 905 and BFTC 909 cell lines were obtained from the German Collection of Microorganisms and Cell Cultures (Braunschweig, Germany). 5637 cells were grown in RPMI 1640 medium, and the other cells were grown in Dulbecco's modified Eagle medium (DMEM). Re-authentication of cells was performed using PowerPlex 16 HS for short tandem repeats analysis at the Johns Hopkins University School of Medicine (JHUSOM), Institute of Genetic Medicine core facility, and all cell lines have been confirmed as authentic. Urine samples from a total 84 UCB subject (24 for training and 60 for validation cohorts) and 207 from population-matched control subjects (24 for training and 183 for validation cohort) were analyzed. These urine samples were obtained from a urinary tract specimen bank maintained within the JHUSOM, Department of Pathology. Control subjects of both the cohorts had no history of genitourinary malignancy. Diagnosis of all UCB specimens was confirmed by a board-certified cytopathologist. Detailed clinicopathological information of UCB cases and controls are provided in Table 1. Thirty human primary UCB and the corresponding adjacent histologically non-cancer urothelial tissue samples were obtained from the, JHUSOM, Department of pathology. Informed consent was obtained from the patients before sample collection. Approval to conduct research on human subjects was obtained from the JHUSOM institutional review boards. This study qualified for exemption under the U.S. Department of Health and Human Services policy for protection of human subjects [45 CFR 46.101(b)].

RNA extraction and quantitative reverse transcriptase polymerase chain reaction (qRT-PCR). Total RNA from cell lines and formaldehyde fixed-paraffin embedded human tissues was isolated using the RNeasy Plus Mini Kit (Qiagen, Valencia, USA) and the RecoverAll™ Total Nucleic Acid Isolation Kit (Ambion, Austin, USA), respectively. Urine samples were centrifuged for 5 min at 1500 rpm and the supernatant was used for RNA extraction as described previously¹⁹. Total RNA extraction from urine was performed using the MirVana miRNA Isolation Kit (Ambion). qRT-PCR was performed using the Fast SYBR Green Master Mix (Thermo Fisher Scientific, Waltham, USA) on a 7900HT Fast Real-Time PCR System (Life technologies, Carlsbad, USA) in triplicate. Primer sequences and the thermal cycling conditions were shown in Table 3. SDS software (Applied Biosystems) was used to determine cycle threshold (Ct) values. Expression levels were quantified relative to β-actin using the 2-ΔΔCt method.

Candidate gene selection to evaluate as a urinary biomarker. To construct a panel of urinary biomarker for cancer detection, 15 potential CSC-related molecules were selected based on our previous findings associated with malignant stemness properties in UCB^{11,19}. A receiver operating characteristic (ROC) analysis was used for evaluating the UCB detection accuracy using urine. ROC analysis method circumvents fluctuations caused by the arbitrarily chosen cutoff value of expression level to differentiate cases and controls as a selection criteria. The optimal cutoff value for distinguishing between UCB and control urine samples was determined using the ROC analysis for each gene. The performance of ROC analysis for each gene was evaluated by the area under the curve (AUC) that is a combined measure of sensitivity and specificity. In addition, the positive and negative likelihood ratio which are not affected by the prevalence of the disease were measured to assess the strength of UCB detection accuracy for each gene.

The Cancer Genome Atlas (TCGA) analysis. The gene expression data of 19 primary UCB samples and the matched tumor adjacent histologically normal samples in the TCGA cohort²⁰ was downloaded from the MethHC database²¹ to determine the expression level of our gene of interest in this external dataset.

Western blotting analysis. Whole cell lysates were extracted using the RIPA buffer (Thermo Scientific) supplemented with 10 μL/mL of the Halt™ Protease Inhibitor Cocktail Kit (Life Technologies) and 30 μL/mL of the Halt™ Phosphatase Inhibitor Cocktail Kit (Life Technologies). CD133 (A3G6K) and ABCG2 (42078) antibodies were obtained from Cell Signaling Technology (Danvers, Mass., USA). YAP1 (ab52771) and CD24 (AF5247-SP) were obtained from Abcam (Cambridge, USA) and R&D Systems (Minneapolis USA) respectively. β-actin (A2228) was obtained from Sigma-Aldrich (St. Louis, USA). Secondary horseradish peroxidase (HRP)—conjugated antibodies were obtained from Cell Signaling Technology. Chemiluminescent detection of HRP-labeled antibodies was performed using Amersham ECL Prime Western Blotting Detection Reagent (GE Healthcare, Piscataway, USA). Expression levels of all candidates were quantified by mylmageAnalysis™ Software (Thermo Scientific) and normalized to β-actin.

Gene silencing. CD24 shRNA Lentiviral Particles (Cat #sc-29978-V) was used for the knockdown of the gene expression (CD24-sh; Santa Cruz Biotechnology, Dallas, USA). shRNA Lentiviral Particles (Cat #sc-108080) was used as a control (CD24-Ctrl; Santa Cruz Biotechnology). Cells were seeded in 24-well plates (5×10⁴ cells per well) for transduction. After 24 hours, lentiviral particles were added to the cells in the presence of 8 mg/mL polybrene (EMD Millipore) and incubated at 37° C. for 4 hours. The medium was then replaced with fresh medium. Stable cells harboring CD24 shRNA were established by antibiotic selection and expression level of CD24 was confirmed by RT-PCR and western blotting in the respective clone.

Sphere formation assay and self-renewal assay. Sphere formation was performed by culturing cells (2×10⁴/well) in DMEM/Ham's F12 50/50 Mix (Mediatech) supplemented with B-27 (Life Technologies), 20 ng/mL of fibroblast growth factors (FGF)-basic (Peprotech, N.J., USA) and 20 ng/mL epidermal growth factor (EGF) (Peprotech). Cell culture was performed in ultra-low attachment 6 well plates (Corning, Lowell, USA) for 14 days. The medium was replaced every other day. Sphere formation was evaluated using the inverted phase-contrast microscope and single sphere with a diameter larger than 100 μm was counted using NIS-Elements Microscope Imaging Software (Nikon Instruments).

For the self-renewal assay, primary spheres were collected by gentle centrifugation (5 min at 400×g), dissociated with Stempro Accutase Cell Dissociation Reagent (Life Technologies), and mechanically disrupted with a pipette. The cell suspension was sieved through 40 μm cell strainer cap filter to achieve a single-cell suspension. Equal numbers of live cells were plated in ultralow attachment plates to generate the second spheres. All the experiments were performed in triplicate and repeated at least three times.

Cell viability assay (MTT assay). Cell viability was measured using the 3-(4, 5-dimethyl thiazol-2-yl)-2, 5-diphenyltetrazolium bromide (MTT) Proliferation Assay Kit (ATCC; Manassas, Va., USA) according to the manufacturer's protocol. Spheroid cells were treated with cisplatin (CDDP, Sigma-Aldrich) for 72 h in ultra-low attachment 96 well plates under serum-free condition. Each assay was performed in triplicate, and each experiment was repeated at least three times.

Invasion assay and wound healing assay. The invasion assay was performed using the 24-well BD BioCoat Matrigel Invasion Chamber (BD Biosciences, San Jose, USA) as described previously²². Cells that had invaded through the membrane were counted under a microscope in 5 randomly selected fields (magnification ×20) per well and averaged.

The wound healing assay was performed using the Culture-Inserts (ibidi, Verona, USA). The area of wound coverage was calculated using NIS-Elements Microscope Imaging Software.

Flow cytometric analysis. For CD24 staining, cells (1×10⁶/100 μL stain buffer) were incubated with PE-conjugated anti-human CD24 antibody (cat #, 560991; BD Biosciences) for 30 min at 4° C. in dark. PE-conjugated IgG2a, κ Isotype (cat #, 555574; BD Biosciences) was used as a control. Data were acquired on a BD FACSCalibur flow cytometer (BD Biosciences) using BD CellQuest Pro software (BD Biosciences).

For the apoptosis assay, the spheroid cells were exposed to CDDP (5 μM) for 72 hours under serum-free medium and stained with PE Annexin V and 7-AAD for discrimination of early and late apoptosis respectively using the PE Annexin V Apoptosis Detection Kit I (BD Biosciences).

In vivo xenograft assay. NOD/SCID/IL2Rγ-/-(NSG) mice were obtained from the JHUSOM animal care facility. All experiments using mice were approved by the JHUSOM Animal Care and Use Committee, and mice were maintained under pathogen-free conditions within the JHUSOM animal care facility in accordance with the American Association of Laboratory Animal Care guidelines. For a limiting-dilution tumor formation assay, serially diluted CD24-sh or CD24-Ctrl spheroid cells (1×10⁴, 1×10³, or 1×10² cells per flank) were suspended in 100 μL of a 1:1 mixture of serum-free DMEM and Cultrex Stem Cell Qualified Reduced Growth Factor Basement Membrane Extract (Trevigen, Gaithersburg, USA), and then injected subcutaneously into both flanks of 4-5 week-old NSG mice. Tumor growth was monitored, and tumor volume was calculated from caliper measurements of two orthogonal diameters [larger (x)] and smaller (y) diameters] using the following formula: volume=xy²/2. The mice were euthanized when tumor reached 2 cm in diameter, or 70 days later.

Preserved patient-derived tumor xenograft (PDX) tissues (CTG1388 and CTG1061) were obtained from Champion Oncology (Maryland, USA). For magnetic-activated cell sorting for CD24, PDX tumors were minced and digested with collagenase type IV (Sigma-Aldrich), hyaluronidase (Sigma-Aldrich), and DNase type IV (Sigma-Aldrich) into HBSS, followed by depletion of red blood cells using ACK lysing buffer (Quality Biological, Gaithersburg, USA). Tumor cells were isolated using Tumor Cell Isolation Kit (Miltenyi Biotec) according to the manufacturer's protocol. Then, tumor cells (1×10⁸) were labelled with PE-conjugated anti-human CD24 antibody (Miltenyi Biotec, Auburn, USA), and subsequently labelled with Anti-PE MultiSort MicroBeads (Miltenyi Biotech). After washing, separation for CD24 negative and positive fraction was performed using MACS Columns and MidiMACS Separator (Miltenyi Biotec) twice. This process leaded to the separation of low CD24 and high CD24 enriched cell population. To confirm the separation, flow cytometric analysis was carried out using PE-conjugated anti-human CD24 antibody (Miltenyi Biotec). PE-conjugated anti-IgG1 κ Isotype (Miltenyi Biotec) was used as controls for CD24 staining.

Statistical analysis. In each set of data analyses, the estimate variation is indicated in each figure as a standard error of mean (SEM). The two groups were compared with the Wilcoxon-Mann-Whitney test for continuous variables and the Fisher's exact test for categorical variables, respectively. The level of statistical significance was set at P<0.05. All statistical analyses were conducted using the JMP 12 software package (SAS Institute).

### Results

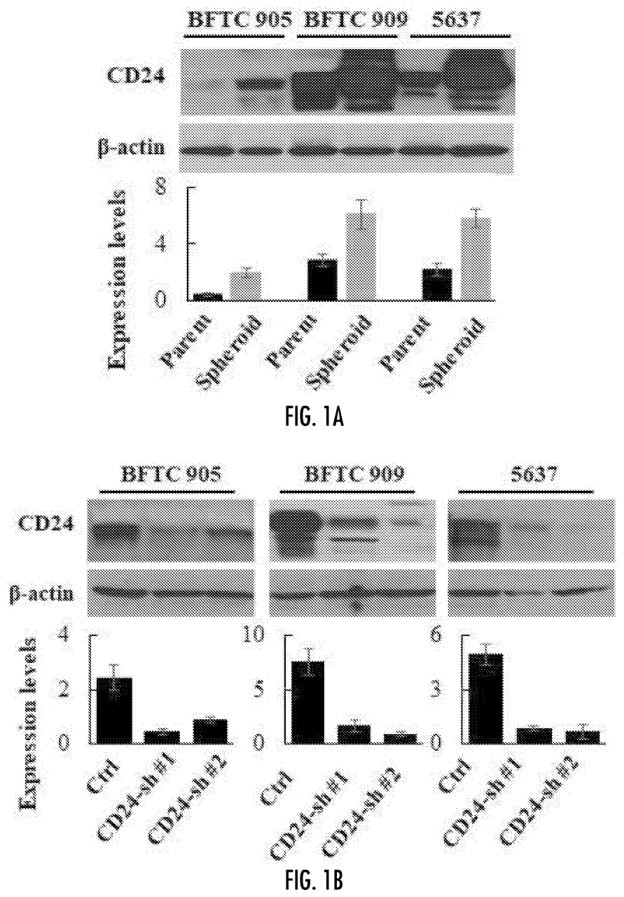

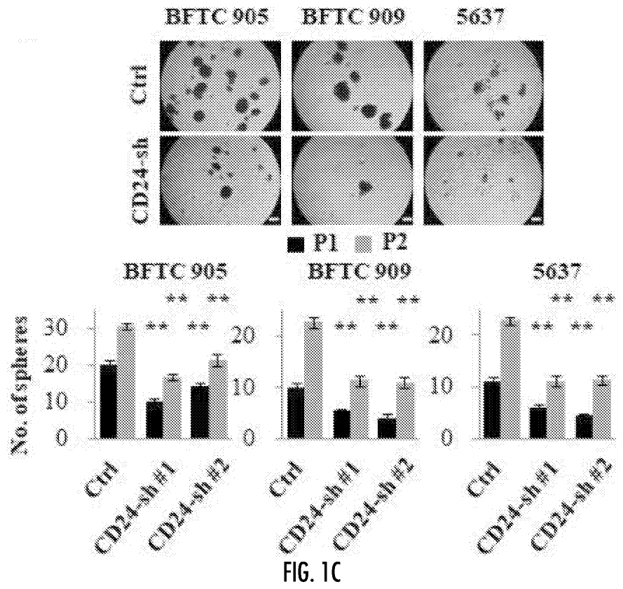

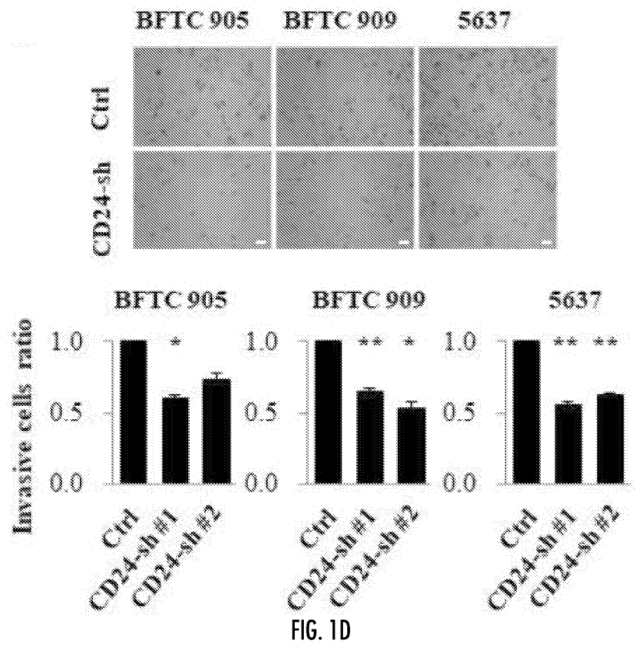

Knockdown of CD24 attenuates urothelial cancer stemness properties. To our knowledge, there is no report whether CD24 functionally contributes to urothelial cancer stem-like traits. As spheroid cells contain enriched stem cell populations 23, we first assessed the expression levels of CD24 in spheroid cells compared with the matched parental cells in BFTC 905, BFTC 909, and 5637 cell lines. The spheroid cells showed higher expression of CD24 than the matched parental cells (FIG. 1A). To determine the functional role of CD24 in urothelial CSCs, a lentiviral-based stable knockdown clones of CD24 (CD24-sh) were established in BFTC 905, BFTC 909, and 5637 cell lines (FIG. 1B). The effect of CD24 knockdown on sphere-forming and self-renewal abilities was analyzed by sphere formation assays. CD24-sh cells generated fewer and smaller spheres compared with the control (CD24-Ctrl) cells through their first and second passages (FIG. 1C). To assess whether spheroid CD24-sh cells sustain an aggressive phenotype comparable to the spheroid CD24-Ctrl cells, we performed invasion assays and found that spheroid CD24-sh cells demonstrated decreased invasion (FIG. 1D).

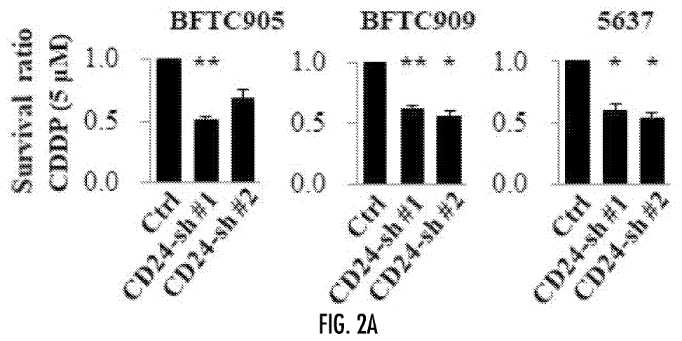

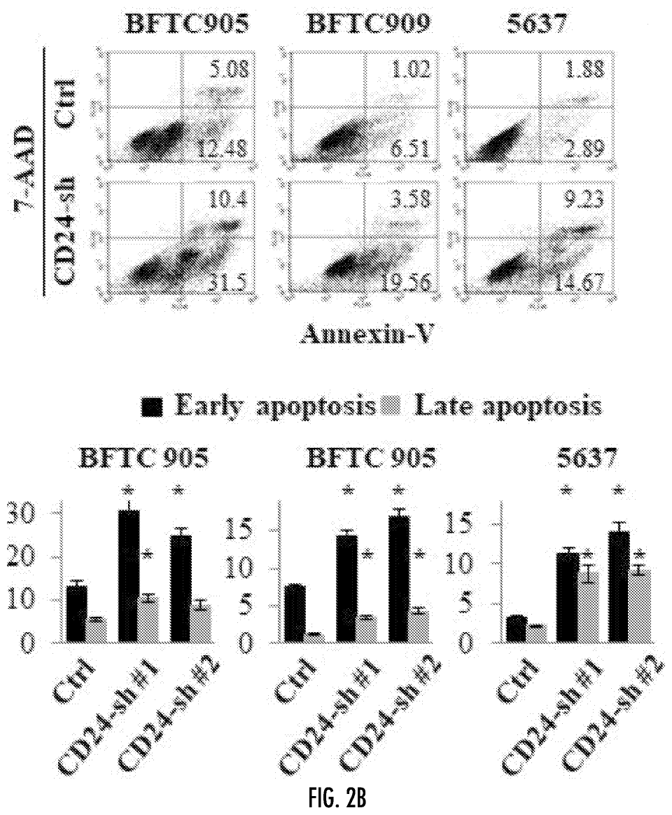

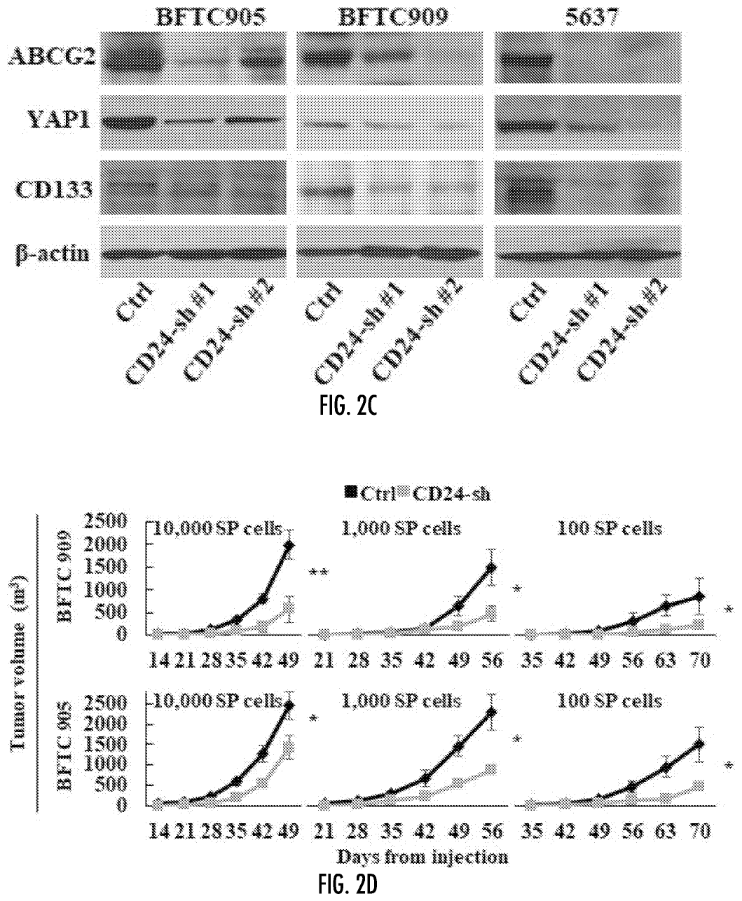

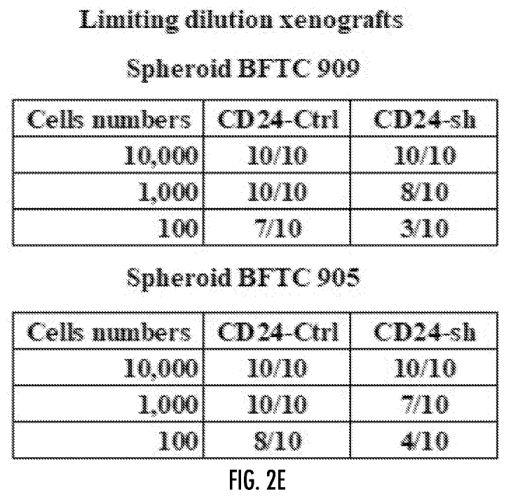

As CSCs are resistant to conventional chemotherapies that efficiently eliminate bulk tumor cells, the viability of spheroid CD24-sh cells was analyzed by treating with cisplatin (CDDP), an important chemotherapeutic agent for the treatment of UCB. As expected, spheroid CD24-sh cells were more sensitive to CDDP treatment than spheroid CD24-Ctrl cells (FIG. 2A). Furthermore, we determined that CD24 knockdown significantly attenuated the anti-apoptotic ability against CDDP treatment in spheroid cells (FIG. 2B).

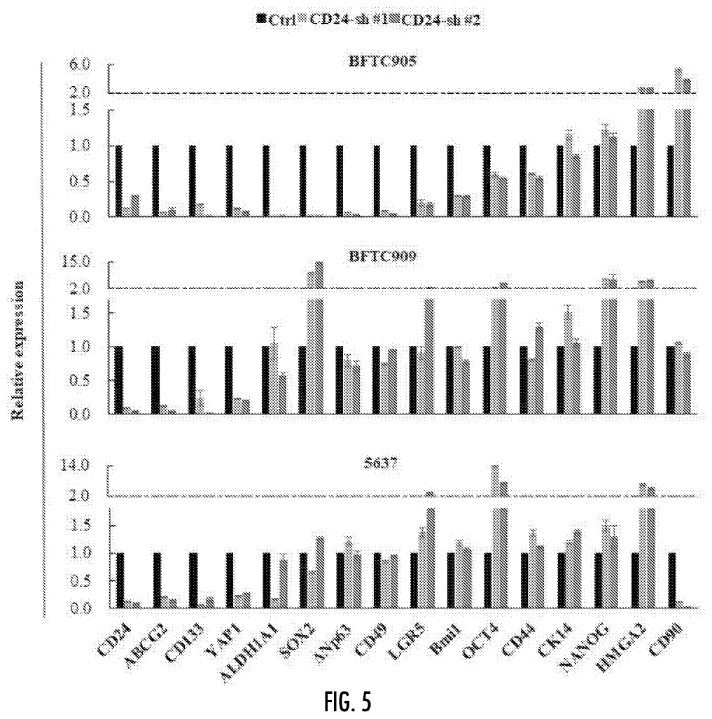

To determine the effect of CD24 knockdown on candidate CSC-related molecules, we tested mRNA expression levels of 15 potential CSC-related molecules in CD24-sh and CD24-Ctrl cells by qRT-PCR. CD24 knockdown led to the downregulation of numerous CSC-related molecules, among which CD133, Yes-associated proteinl (YAP1), and the drug efflux transporter ATP-binding cassette subfamily G member 2 (ABCG2) were found to be consistently altered due to loss of CD24 in the three UCB cell lines (FIG. 5). We confirmed similar findings at the protein level (FIG. 2C).

Knockdown of CD24 attenuates in vivo tumorigenicity. Since an important feature of CSCs is efficient in vivo tumorigenesis at a limiting-dilution xenograft 24, we performed the tumor formation assays with serial dilutions of spheroid CD24-sh cells and CD24-Ctrl cells. Serially diluted spheroid CD24-sh or CD24-Ctrl cells (1×104, 1×103, or 1×102 cells per flank) were injected subcutaneously into both flanks of NSG mice (five mice per group). Comparable with our in vitro assays data, the spheroid CD24-sh cells had significantly reduced tumor growth compared with the spheroid CD24-Ctrl cells (FIG. 2D). The spheroid CD24-Ctrl cells were sufficient for tumor development by injecting as few as 100 cells into NSG mice. Seven and 8 tumors were developed in a total of 10 flanks of 5 mice injected with spheroid CD24-Ctrl BFTC 909 and CD24-Ctrl BFTC 905 cells respectively, within a 70 day follow-up period after cell injection. In contrast, the spheroid CD24-sh cells showed decreased tumor initiation frequency (development of 3 and 4 tumors per 10 flanks of mice for CD24-sh BFTC 909 and CD24-sh BFTC 905 cells, respectively; FIG. 2E).

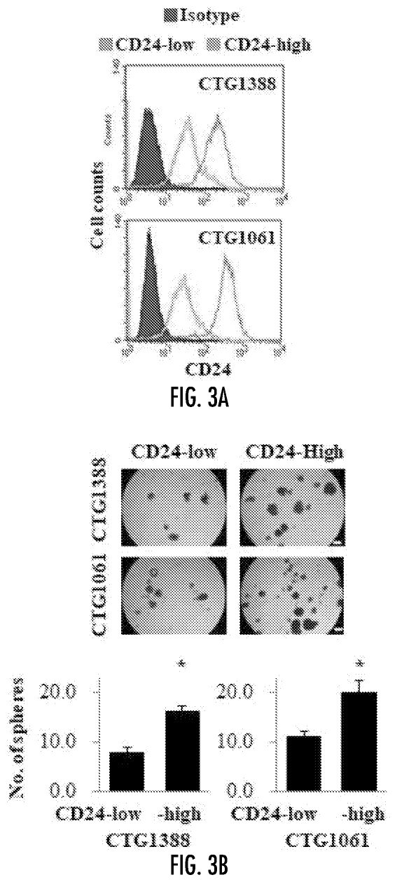

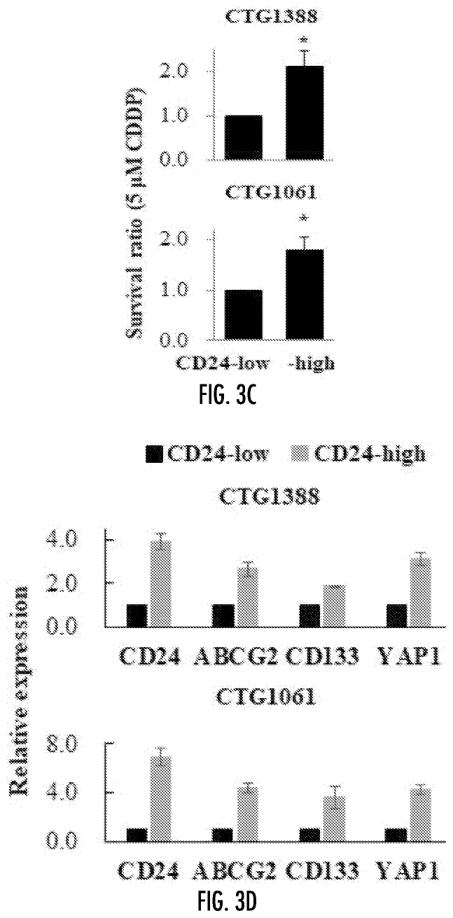

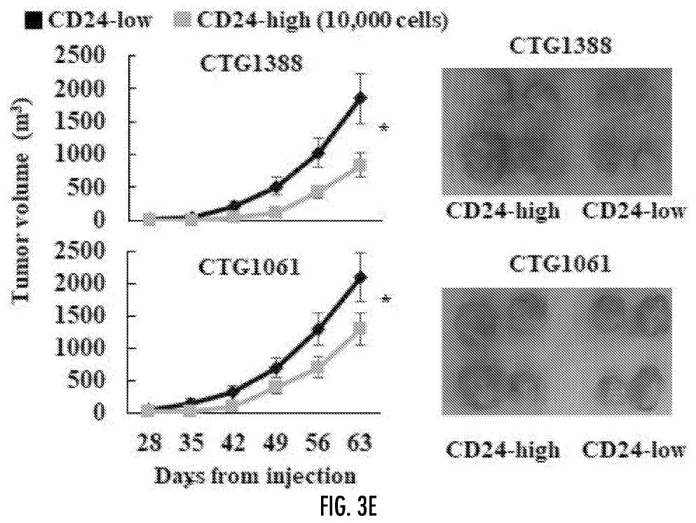

CD24-expressing cells isolated from patient-derived xenograft (PDX) models exhibit enhanced cancer stem-like traits. To test whether cells with high endogenous CD24 expression possess enhanced stemness properties, we isolated high- and low-CD24-expressing cells from two PDX models using the magnetic-activated cell sorting approach. One model was established from a primary site (CTG1388) and a second model was established from a metastatic site (CTG1061). We confirmed the expression status of CD24 by flow cytometric analysis (FIG. 3A). The high-CD24-expressing cells exhibited greater sphere-forming and chemo-resistant abilities than the low-CD24-expressing cells (FIG. 3B-C). In addition, the link of CD24 with CD133, YAP1, and ABCG2 was solidified by our observation of high expression of these molecules in PDX-derived high-CD24-expressing cells, as compared to low-CD24-expressing cells (FIG. 3D). Furthermore, the high-CD24-expressing cells grew faster and generated larger tumors than the low-CD24-expressing cells after subcutaneous injection of 1×104 cells per flank into NSG mice (FIG. 3E). Collectively, our findings suggest a crucial role of CD24 in urothelial cancer stem-like traits.

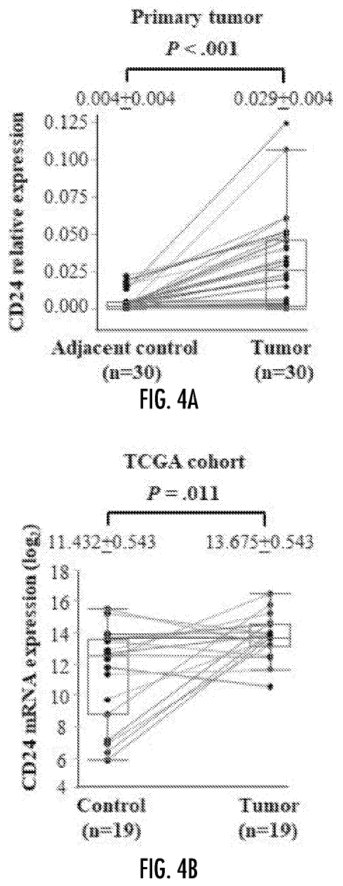

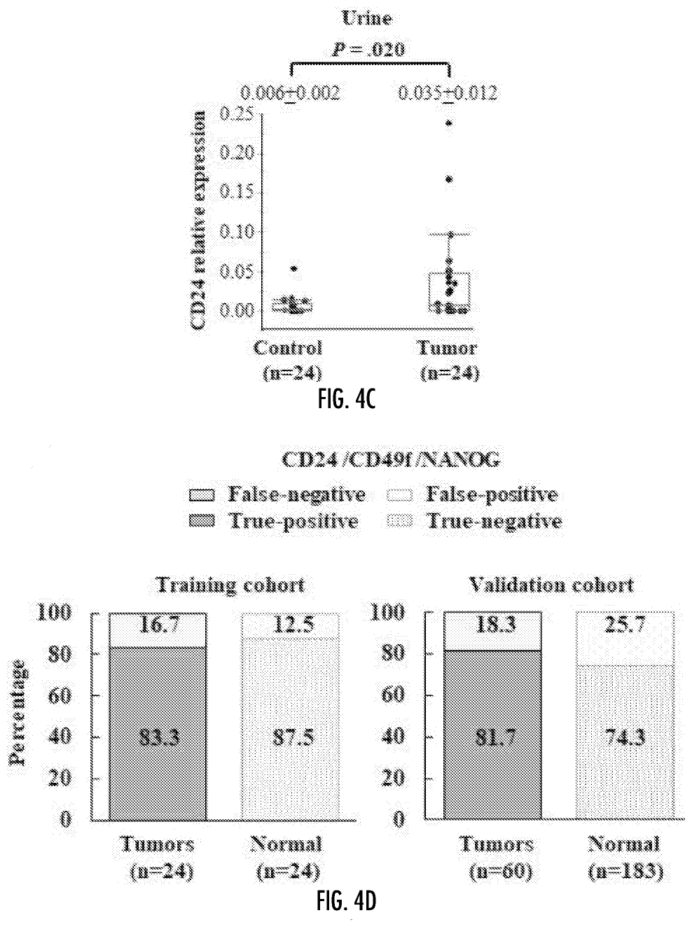

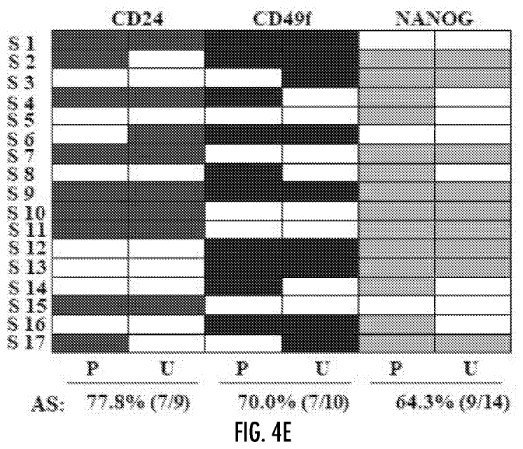

CD24 has potential as a urinary biomarker for UCB detection. To test the cancer specificity of CD24 expression in primary UCB, we analyzed mRNA expression levels of 30 primary UCB and matched adjacent histologically non-cancer tissues by qRT-PCR. Consistent with previous reports 12-14, CD24 showed significantly higher expression in primary tumors compared with the corresponding adjacent normal tissues (FIG. 4A). Furthermore, analysis of the TCGA UCB cohort generated similar findings in tumor and the matched adjacent normal tissues (FIG. 4B).

The clinical utility of CD24 as a biomarker for cancer detection has not been determined. Given the cancer-specific elevation of CD24 expression in primary tumors, we next assessed the potential for non-invasive cancer detection using a total of 48 urine samples (24 UCB and 24 control subjects) as a training cohort (Table 1). The expression level of CD24 in urine was significantly higher in UCB subjects than in controls (FIG. 4C). The optimal cutoff value for distinguishing between urine samples from UCB and control subjects was calculated using a receiver operating characteristic (ROC) analysis. By using the optimal cutoff value of CD24 expression, the sensitivity and specificity of CD24 for cancer detection were 45.8% and 95.8% respectively (Table 4). The high specificity indicates that CD24 may be a potential urinary biomarker for UCB detection. We hypothesized that the low sensitivity was due to heterogeneity among UCB and that the addition of other markers could improve sensitivity.

We previously reported SOX2 as a potential urine-based biomarker for non-invasive early detection of UCB 19, and this molecule is an established regulator of CSCs and play a considerable role in tumor initiation 9. To further identify CSC-associated urine-based biomarkers, we tested 15 CSC-related molecules by candidate gene approach in urine from 24 UCB and 24 controls subjects (total 48 urine samples as a training cohort). Among these 15 molecules, NANOG, CD49f, LGR5, ΔNp63, SOX2 and CD24 showed significantly higher expression levels in urine from UCB subjects compared with control samples (Table 4). By determining the optimal cutoff using ROC curves for each molecule, the individual sensitivity and specificity of these six molecules (NANOG, CD49f, LGR5, ΔNp63, SOX2 and CD24) for cancer detection ranged from 29.2% to 62.5% and 83.3% to 100%, respectively (Table 4). We further assessed the expression pattern spectrum of these six molecules. When the positive expression of at least one of the six molecules was considered, the sensitivity was 95.8%, while the specificity decreased to 50.0% (Table 5). When combination of CD24, CD49f, and NANOG was considered, a high UCB detection accuracy was achieved, with a sensitivity of 83.3% and specificity of 87.5% (FIG. 4D and Table 2).

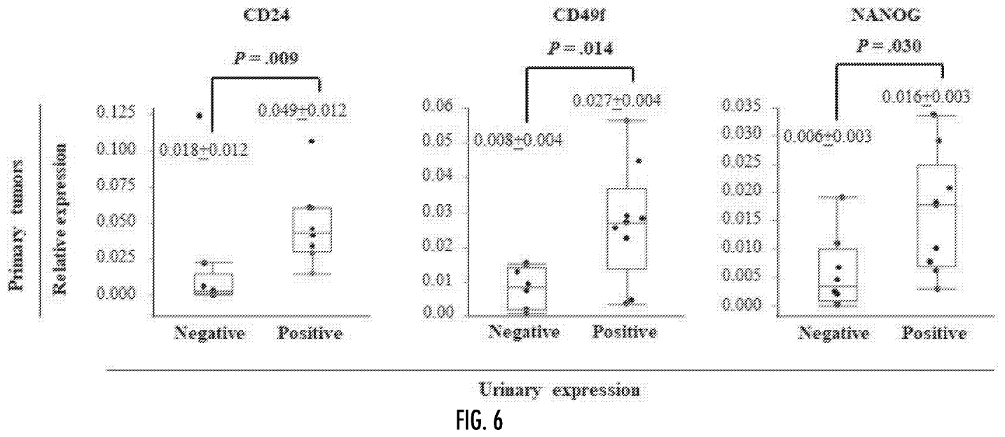

To determine the analytical sensitivity, we analyzed expression of later three molecules (CD24, CD49f, and NANOG) in 17 primary UCB tissues with matched urine samples. The expression levels of these three molecules (CD24, CD49f, and NANOG) in primary tumor tissues were significantly higher in subjects with positive expression in urine samples than in those with negative urine expression (FIG. 6). The concordance rate between primary tumors and the matched urine samples (analytical sensitivity) was 77.8% (7/9) for CD24, 70.0% (7/10) for CD49f, and 64.3% (9/14) for NANOG (FIG. 4E). The clinical sensitivity (detection of cancer by urine test of these 3 genes) of CD24, CD49f and NANOG were 47.1% (8/17), 52.93% (9/17) and 52.93% (9/17) respectively (FIG. 4E). At least one of the 3 genes was overexpressed in primary tumors and urines of all the samples analyzed. Based on reasonable analytical and clinical sensitivity in the training cohort, a combination panel of these three CSC-related molecules may have potential to detect UCB with high sensitivity and specificity using clinical samples.

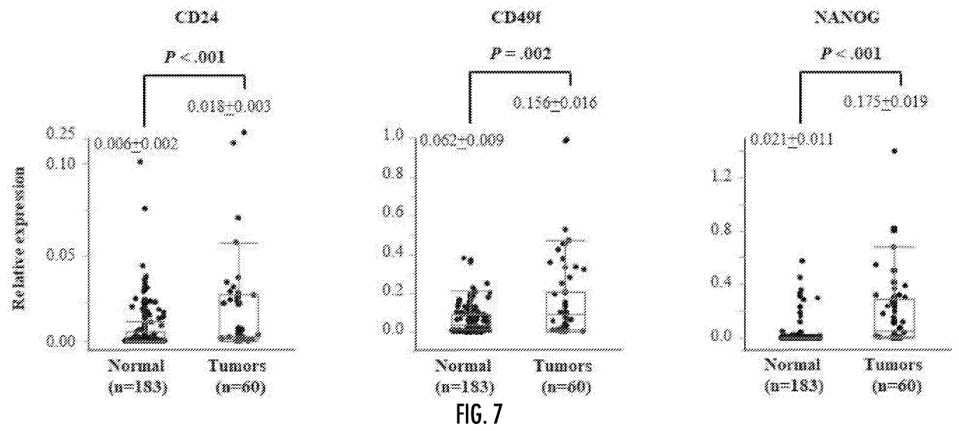

Validation of a panel of three CSC-related genes (CD24, CD49f, and NANOG) in an independent cohort of urine sample for early detection of UCB. To confirm the detection accuracy of combination of three CSC-related molecules (CD24, CD49f, and NANOG), we tested an independent validation cohort consisting of urine samples from 60 UCB and 183 control subjects (Table 1). Again, higher expression levels of these three molecules were observed in the urine samples from UCB subjects than controls (FIG. 7). Using the same cutoff as of training cohort, the individual sensitivity and specificity for cancer detection were 35.0% (21/60) and 91.3% (167/183) for CD24, 35.0% (21/60) and 83.6% (153/183) for CD49f, and 51.7% (31/60) and 88.5% (162/183) for NANOG, respectively (Table 2). The combination panel of these 3 genes yielded an overall sensitivity of 81.7% (49/60) and specificity of 74.3% (136/183) (FIG. 4D). Of note, this combination panel (CD24, CD49f and NANOG) detected NMIBC with a sensitivity of 80.9% (38/47) and low-grade UCB with a sensitivity of 80.0% (12/15) (Table 2). In addition, 14 (82.4%) out of a total of 17 cytology negative UCB samples were detected by this combination panel.

Finally, we assessed the UCB detection accuracy by 3 markers (CD24, CD49f and NANOG) panel urine testing by combining training and validation cohorts (total 84 and 207 urine samples from UCB and control subjects, respectively). This analysis yielded a sensitivity and specificity of 82.1% and 75.8%, respectively (Table 2). The positive and negative likelihood ratio of this test was 3.40 (95% CI, 2.71-4.08) and 0.24 (95% CI, 0.15-0.36), respectively (Table 6).

### Discussion

A growing body of evidence indicates that CSCs are a driving force behind tumor initiation, metastasis, and therapeutic resistance¹⁰. Therefore, identification of the molecules responsible for urothelial cancer sternness properties may facilitate the development of novel therapeutic strategies and biomarkers for non-invasive UCB detection. Here, we showed that CD24 plays an essential role in maintaining the urothelial cancer stem-like traits. In addition, to our knowledge, we are reporting for the first time that CSC-related molecules have potential as clinically useful urinary biomarkers for non-invasive detection of UCB.

CD24 is a lynchpin of tumorigenesis and metastatic progression in UCB^{13,15,16}. However, the relevance of CD24 in urothelial cancer stem-like traits remains unclear. In this study, for the first time we characterized CD24 as a major determinant of urothelial sternness, supporting previous findings that CD24-expressing cells exhibit aggressive phenotype. Although we genetically inhibited CD24 using a lentiviral-based approach that may not be suitable for clinical use, Overdevest et al. demonstrated that treatment with an anti-CD24 monoclonal antibody led to reduced tumor growth and metastasis, resulting in prolonged survival in UCB xenograft model¹⁵. Collectively, our pre-clinical data suggests that CD24 could be a promising therapeutic target to efficiently eliminate urothelial CSCs.

The exact molecular mechanisms for CSC generation and maintenance via CD24 are incompletely understood. We observed the downregulation of several CSC-related molecules such as CD133, ABCG2, and YAP1 in CD24-sh cells and expression level of these molecules were higher in high-CD24-expressing cells, indicating a potential crosstalk between CD24 and these CSC-related molecules. CD133, a pentaspan transmembrane glycoprotein, has been used as a surface marker for isolation of urothelial CSCs^{11,25}. ABCG2 is a drug transporter and this molecule actively effluxes varieties of chemotherapeutic agents that may provide CSCs with a selective survival advantage against chemotherapy²⁶. YAP1 is a downstream transcription effector of the Hippo pathway²⁷ and we recently demonstrated that urothelial cancer stem-like traits are driven by the YAP1-SOX2 signaling axis that is an upstream regulator of CD24 expression¹¹. CD24 interacts with Src to promote its kinase activity 17, and activated Src has been implicated in regulating YAP1^{11,28}. Thus, the regulatory circuitry between YAP1 and CD24 may accelerate urothelial CSC maintenance and progression. Further research is needed to elucidate these complex crosstalk mechanisms.

Urine cytology analysis is a non-invasive approach for cancer detection with a high specificity (>95%)²⁹, but it is limited by its low sensitivity (35-55%)⁵, especially for low-grade (<20%) and low-stage (<40%) disease^{6,7}. Sensitivity is generally considered more important than specificity for screening and surveillance, as the missing of early disease increases the risk of progression to advanced disease and a poor clinical outcome³⁰. Although several urine-based diagnostic assays have been approved by the U.S. Food and Drug Administration (FDA)^{5,31,32}, these assays do not overcome the low sensitivity for low-grade disease. Therefore, improvement of the detection sensitivity for low-stage and low-grade disease is one of the central goals of urine-based tests. In this study, the combination panel (CD24, CD49f, and NANOG) yielded high sensitivity for cancer detection not only for NMIBC (80.9%), but also for low-grade UCB (80.0%). Most importantly, 82.4% of UCB specimens negative by cytology were positive by CSC marker test in urine. Therefore, this CSC-related panel with high sensitivity could provide a valuable adjunct to urine cytology for UCB detection.

CD49f mediates the stem cell niche via interactions with the extracellular matrix and communication between tumor cells and the tumor-microenvironment³³. In UCB, CD49f is down-regulated during differentiation³⁴ and has been utilized to enrich CSC population³⁵. NANOG is a key pluripotent transcription factor³⁶ and predominantly expressed in urothelial CSCs³⁷. Thus, all the three molecules (CD24, CD49f and NANOG) act as biologically relevant CSC factors in UCB. Because we did not observe concordant changes in CD49f and NANOG expression due to alteration of CD24 expression in CD24-sh cells and CD24-expressing cells, the combination panel may be able to detect different CSC populations and each of these molecules could be an independent marker for this heterogeneous disease. In fact, 39 of 60 UCB urine samples in the validation cohort showed negative expression of CD24, and 9 (23.1%) and 21 (53.8%) out of 39 urine samples without CD24 expression showed positive for CD49f and NANOG, respectively. This observation also supports the multi-clonal origin of this heterogeneous disease³⁸. Although its needs further validation, some in consistency of findings between primary tumors and urine may be due to the site of the primary tumors that were analyzed. It could happen that we analyzed the tumor site that did not shed the tumor cells in the urine and the source of tumor cell may be other sites of the same bladder.

Several studies reported promising panels of urinary mRNAs determined by qRT-PCR for UCB detection, including the commercially available Cxbladder assay³⁹⁻⁴². Compared with these assays, overall sensitivity of our assay was similar, but specificity was relatively low, partially due to the use of different reference genes and selection approaches. Cxbladder adapted CXCR2 as a reference gene to reduce the false positive rate³⁹. Other assays considered genes that are expressed stably and with little variability in exfoliated urinary cells⁴⁰⁻⁴². Thus, the use of suitable reference genes may improve the specificity of our assay. In addition, the novel transcriptome profiling approach may yield more sensitive and specific CSC-related biomarkers in urine⁴³.

Study limitations include the relatively small sample size and possible bias due to retrospective analysis and empirical selection of CSC-related molecules for the urinary biomarker. In addition, our cohort may not be representative of the general population at risk for UCB because of the lack of relevant clinical data, including smoking history, occupational exposure history, and patient outcome. Infections or any other inflammatory disease in the urinary tract may influence expression of these biomarkers. Therefore, while promising, our findings cannot be considered conclusive, and extensive validation is needed in a larger independent cohort including various urologic conditions to assess the clinical utility of combination panel of three CSC-related molecules.

In summary, we demonstrated that CD24 drives cancer stem-like traits and serves as a promising non-invasive urinary biomarker for UCB detection. In addition, we also identified a panel of CSC-related molecules that has potential as a urinary biomarker for UCB detection with a high sensitivity and specificity. These findings may facilitate the development of improved therapeutic strategies and non-invasive detection of UCB.

### REFERENCES

1. Siegel RL, Miller KD, Jemal A. Cancer Statistics, 2017. CA: a cancer journal for clinicians 2017; 67(1): 7-30; e-pub ahead of print 2017/01/06; doi 10.3322/caac.21387.

2. Hall MC, Chang SS, Dalbagni G, Pruthi RS, Seigne JD, Skinner EC et al. Guideline for the management of nonmuscle invasive bladder cancer (stages Ta, T1, and Tis): 2007 update. The Journal of urology 2007; 178(6): 2314-2330; e-pub ahead of print 2007/11/13; doi 10.1016/j.juro.2007.09.003.

3. Aldousari S, Kassouf W. Update on the management of non-muscle invasive bladder cancer. Can Urol Assoc J 2010; 4(1): 56-64; e-pub ahead of print 2010/02/19.

4. Knowles MA, Hurst CD. Molecular biology of bladder cancer: new insights into pathogenesis and clinical diversity. Nature reviews Cancer 2015; 15(1): 25-41; doi 10.1038/nrc3817.

5. Schmitz-Drager BJ, Droller M, Lokeshwar VB, Lotan Y, Hudson MA, van Rhijn BW et al. Molecular markers for bladder cancer screening, early diagnosis, and surveillance: the WHO/ICUD consensus. Urol Int 2015; 94(1): 1-24; e-pub ahead of print 2014/12/17; doi 10.1159/000369357.

6. Lokeshwar VB, Habuchi T, Grossman HB, Murphy WM, Hautmann SH, Hemstreet GP, 3rd et al. Bladder tumor markers beyond cytology: International Consensus Panel on bladder tumor markers. Urology 2005; 66(6 Suppl 1): 35-63; e-pub ahead of print 2006/01/10; doi 10.1016/j.urology.2005.08.064.

7. Fantony JJ, Inman BA. It May Be Time to Abandon Urine Tests for Bladder Cancer. J Natl Compr Canc Netw 2015; 13(9): 1163-1166; e-pub ahead of print 2015/09/12.

8. Zhang ML, Rosenthal DL, VandenBussche CJ. The cytomorphological features of low-grade urothelial neoplasms vary by specimen type. Cancer Cytopathol 2016; 124(8): 552-564; e-pub ahead of print 2016/03/29; doi 10.1002/cncy.21716.

9. Ho PL, Kurtova A, Chan KS. Normal and neoplastic urothelial stem cells: getting to the root of the problem. Nat Rev Urol 2012; 9(10): 583-594; e-pub ahead of print 2012/08/15; doi 10.1038/nruro1.2012.142.

10. Beck B, Blanpain C. Unravelling cancer stem cell potential. Nature reviews Cancer 2013; 13(10): 727-738; e-pub ahead of print 2013/09/26; doi 10.1038/nrc3597.

11. Ooki A, Del Carmen Rodriguez Pena M, Marchionni L, Dinalankara W, Begum A, Hahn NM et al. YAP1 and COX2 Coordinately Regulate Urothelial Cancer Stem-like Cells. Cancer research 2018; 78(1): 168-181; e-pub ahead of print 2017/11/29; doi 10.1158/0008-5472.can-17-0836.

12. Lee JH, Kim SH, Lee ES, Kim YS. CD24 overexpression in cancer development and progression: a meta-analysis. Oncol Rep 2009; 22(5): 1149-1156; e-pub ahead of print 2009/09/30.

13. Smith SC, Oxford G, Wu Z, Nitz MD, Conaway M, Frierson HF et al. The metastasis-associated gene CD24 is regulated by Ral GTPase and is a mediator of cell proliferation and survival in human cancer. Cancer research 2006; 66(4): 1917-1922; e-pub ahead of print 2006/02/21; doi 10.1158/0008-5472. can-05-3855.

14. Agarwal N, Dancik GM, Goodspeed A, Costello JC, Owens C, Duex JE et al. GON4L Drives Cancer Growth through a YY1-Androgen Receptor-CD24 Axis. Cancer research 2016; 76(17): 5175-5185; e-pub ahead of print 2016/06/18; doi 10.1158/0008-5472.can-16-1099.

15. Overdevest JB, Thomas S, Kristiansen G, Hansel DE, Smith SC, Theodorescu D. CD24 offers a therapeutic target for control of bladder cancer metastasis based on a requirement for lung colonization. Cancer research 2011; 71(11): 3802-3811; e-pub ahead of print 2011/04/13; doi 10.1158/0008-5472.can-11-0519.

16. Overdevest JB, Knubel KH, Duex JE, Thomas S, Nitz MD, Harding MA et al. CD24 expression is important in male urothelial tumorigenesis and metastasis in mice and is androgen regulated. Proc Natl Acad Sci U S A 2012; 109(51): E3588-3596; e-pub ahead of print 2012/09/27; doi 10.1073/pnas.1113960109.

17. Lee TK, Castilho A, Cheung VC, Tang KH, Ma S, Ng IO. CD24(+) liver tumor-initiating cells drive self-renewal and tumor initiation through STAT3-mediated NANOG regulation. Cell stem cell 2011; 9(1): 50-63; e-pub ahead of print 2011/07/06; doi 10.1016/j.stem.2011.06.005.

18. Naumov I, Zilberberg A, Shapira S, Avivi D, Kazanov D, Rosin-Arbesfeld R et al. CD24 knockout prevents colorectal cancer in chemically induced colon carcinogenesis and in APC(Min)/CD24 double knockout transgenic mice. Int J Cancer 2014; 135(5): 1048-1059; e-pub ahead of print 2014/02/07; doi 10.1002/ijc.28762.

19. Ooki A, Begum A, Marchionni L, VandenBussche CJ, Mao S, Max K et al. Arsenic Promotes the COX2/PGE2-50X2 Axis to Increase the Malignant Stemness Properties of Urothelial Cells. Int J Cancer 2018; e-pub ahead of print 2018/02/06; doi 10.1002/ijc.31290.

20. Network. CGAR. Comprehensive molecular characterization of urothelial bladder carcinoma. Nature 2014; 507(7492): 315-322; e-pub ahead of print 2014/01/31; doi 10.1038/nature12965.

21. Huang WY, Hsu SD, Huang HY, Sun YM, Chou CH, Weng SL et al. MethHC: a database of DNA methylation and gene expression in human cancer. Nucleic Acids Res 2015; 43(Database issue): D856-861; e-pub ahead of print 2014/11/16; doi 10.1093/nar/gku1151.

22. Ooki A, Yamashita K, Kikuchi S, Sakuramoto S, Katada N, Kokubo K et al. Potential utility of HOP homeobox gene promoter methylation as a marker of tumor aggressiveness in gastric cancer. Oncogene 2010; 29(22): 3263-3275; e-pub ahead of print 2010/03/17; doi 10.1038/onc.2010.76.

23. Pastrana E, Silva-Vargas V, Doetsch F. Eyes wide open: a critical review of sphere-formation as an assay for stem cells. Cell stem cell 2011; 8(5): 486-498; e-pub ahead of print 2011/05/10; doi 10.1016/j.stem.2011.04.007.

24. Clarke MF, Dick JE, Dirks PB, Eaves CJ, Jamieson CH, Jones DL et al. Cancer stem cells—perspectives on current status and future directions: AACR Workshop on cancer stem cells. Cancer research 2006; 66(19): 9339-9344; e-pub ahead of print 2006/09/23; doi 10.1158/0008-5472. can-06-3126.

25. Bentivegna A, Conconi D, Panzeri E, Sala E, Bovo G, Vigano P et al. Biological heterogeneity of putative bladder cancer stem-like cell populations from human bladder transitional cell carcinoma samples. Cancer Sci 2010; 101(2): 416-424; e-pub ahead of print 2009/12/08; doi 10.1111/j.1349-7006.2009.01414.x.

26. Patrawala L, Calhoun T, Schneider-Broussard R, Zhou J, Claypool K, Tang DG. Side population is enriched in tumorigenic, stem-like cancer cells, whereas ABCG2+ and ABCG2− cancer cells are similarly tumorigenic. Cancer research 2005; 65(14): 6207-6219; e-pub ahead of print 2005/07/19; doi 10.1158/0008-5472.can-05-0592.

27. Zanconato F, Cordenonsi M, Piccolo S. YAP/TAZ at the Roots of Cancer. Cancer Cell 2016; 29(6): 783-803; doi 10.1016/j.cce11.2016.05.005.

28. Taniguchi K, Wu LW, Grivennikov SI, de Jong PR, Lian I, Yu FX et al. A gp130-Src-YAP module links inflammation to epithelial regeneration. Nature 2015; 519(7541): 57-62; e-pub ahead of print 2015/03/04; doi 10.1038/nature14228.

29. Glas AS, Roos D, Deutekom M, Zwinderman AH, Bossuyt PM, Kurth KH. Tumor markers in the diagnosis of primary bladder cancer. A systematic review. The Journal of urology 2003; 169(6): 1975-1982; e-pub ahead of print 2003/05/29; doi 10.1097/01.ju.0000067461.30468.6d.

30. Sanchez-Ortiz RF, Huang WC, Mick R, Van Arsdalen KN, Wein AJ, Malkowicz SB. An interval longer than 12 weeks between the diagnosis of muscle invasion and cystectomy is associated with worse outcome in bladder carcinoma. The Journal of urology 2003; 169(1): 110-115; discussion 115; e-pub ahead of print 2002/12/13; doi 10.1097/01.ju.0000039620.76907.0d.

31. Chou R, Gore JL, Buckley D, Fu R, Gustafson K, Griffin JC et al. Urinary Biomarkers for Diagnosis of Bladder Cancer: A Systematic Review and Meta-analysis. Ann Intern Med 2015; 163(12): 922-931; e-pub ahead of print 2015/10/27; doi 10.7326/m15-0997.

32. Mbeutcha A, Lucca I, Mathieu R, Lotan Y, Shariat SF. Current Status of Urinary Biomarkers for Detection and Surveillance of Bladder Cancer. Urol Clin North Am 2016; 43(1): 47-62; e-pub ahead of print 2015/11/29; doi 10.1016/j.uc1.2015.08.005.

33. Krebsbach PH, Villa-Diaz LG. The Role of Integrin alpha6 (CD49f) in Stem Cells: More than a Conserved Biomarker. Stem Cells Dev 2017; 26(15): 1090-1099; e-pub ahead of print 2017/05/13; doi 10.1089/scd.2016.0319.

34. Volkmer JP, Sahoo D, Chin RK, Ho PL, Tang C, Kurtova AV et al. Three differentiation states risk-stratify bladder cancer into distinct subtypes. Proc Natl Acad Sci USA 2012; 109(6): 2078-2083; e-pub ahead of print 2012/02/07; doi 10.1073/pnas.1120605109.

35. Li DR, Zhang H, Peek E, Wang S, Du L, Li G et al. Synergy of Histone-Deacetylase Inhibitor AR-42 with Cisplatin in Bladder Cancer. The Journal of urology 2015; 194(2): 547-555; e-pub ahead of print 2015/03/10; doi 10.1016/j.juro.2015.02.2918.

36. Gong S, Li Q, Jeter CR, Fan Q, Tang DG, Liu B. Regulation of NANOG in cancer cells. Mol Carcinog 2015; 54(9): 679-687; e-pub ahead of print 2015/05/28; doi 10.1002/mc.22340.

37. Zhang Y, Wang Z, Yu J, Shi J, Wang C, Fu W et al. Cancer stem-like cells contribute to cisplatin resistance and progression in bladder cancer. Cancer letters 2012; 322(1): 70-77; e-pub ahead of print 2012/02/22; doi 10.1016/j.canlet.2012.02.010.

38. Paiss T, Wohr G, Hautmann RE, Mattfeldt T, Muller M, Haeussler J et al. Some tumors of the bladder are polyclonal in origin. J Urol 2002; 167: 718-723.

39. Holyoake A, O'Sullivan P, Pollock R, Best T, Watanabe J, Kajita Y et al. Development of a multiplex RNA urine test for the detection and stratification of transitional cell carcinoma of the bladder. Clin Cancer Res 2008; 14(3): 742-749; e-pub ahead of print 2008/02/05; doi 10.1158/1078-0432.ccr-07-1672.

40. Ribal MJ, Mengual L, Lozano JJ, Ingelmo-Torres M, Palou J, Rodriguez-Faba O et al. Gene expression test for the non-invasive diagnosis of bladder cancer: A prospective, blinded, international and multicenter validation study. Eur J Cancer 2016; 54: 131-138; e-pub ahead of print 2016/01/14; doi 10.1016/j.ejca.2015.11.003.

41. Urquidi V, Netherton M, Gomes-Giacoia E, Serie D, Eckel-Passow J, Rosser CJ et al. Urinary mRNA biomarker panel for the detection of urothelial carcinoma. Oncotarget 2016; 7(25): 38731-38740; e-pub ahead of print 2016/10/23; doi 10.18632/oncotarget.9587.

42. Sin MLY, Mach KE, Sinha R, Wu F, Trivedi DR, Altobelli E et al. Deep Sequencing of Urinary RNAs for Bladder Cancer Molecular Diagnostics. Clin Cancer Res 2017; 23(14): 3700-3710; e-pub ahead of print 2017/02/15; doi 10.1158/1078-0432.ccr-16-2610.

43. Cieslik M, Chinnaiyan AM. Cancer transcriptome profiling at the juncture of clinical translation. Nature reviews Genetics 2017; e-pub ahead of print 2017/12/28; doi 10.1038/nrg.2017.96.

TABLE 1
The clinicopathological features of urine cohorts in this study

| | Samples | | | |
|---|---|---|---|---|
| | Training cohort | | Validation cohort | |
| | Tumor | Control | Tumor | Control |
| Patients | (n = 24) | (n = 24) | (n = 60) | (n = 183) |
| Age (years) | | | | |
| Mean ± SEM (years) | 71.29 ± 2.13 | 62.79 ± 2.56 | 62.87 ± 1.25 | 63.21 ± 1.19 |
| Median (years) | 71 | 64 | 63 | 66 |
| Range (years) | 54-90 | 26-81 | 46-83 | 21-92 |
| Race | | | | |
| White | 17 | 15 | 25 | 128 |
| Black | 4 | 8 | 4 | 42 |
| Othres | 2 | 1 | 0 | 13 |
| Unkonwn | 1 | 0 | 31 | 0 |
| Gender | | | | |
| Female | 7 | 6 | 15 | 55 |
| Male | 17 | 18 | 39 | 128 |
| Unkonwn | 0 | 0 | 6 | 0 |
| Histological grade | | | | |
| High | 17 | N/A | 38 | N/A |
| Low | 6 | N/A | 15 | N/A |
| Unkonwn | 1 | N/A | 7 | N/A |
| Invasion of the muscularis propria | | | | |
| NMIBC | 14 | N/A | 47 | N/A |
| MIBC | 0 | N/A | 9 | N/A |
| Unkonwn | 10 | N/A | 4 | N/A |
| Cytology | | | | |
| Negative^{a} | 16 | N/A | 17 | N/A |
| Positive | 8 | N/A | 40 | N/A |
| Unkonwn | 0 | N/A | 3 | N/A |

Abbreviations:
N/A, not applicable;
NMIBC, non-muscle invasive bladder cancer;
MIBC, muscle invasive bladder cancer.
^{a}Negative cytology includes atypical urothelial cells and suspicious urothelial cancer cells

TABLE 2
The bladder cancer detection accuracy of a panel of 3 genes in urine samples

| | CD24 | | | NANOG | | | CD49f | | | CD24/NANOG/CD49f | | |
|---|---|---|---|---|---|---|---|---|---|---|---|---|
| Characteristics | Sensitivity | | Specificity | Sensitivity | | Specificity | Sensitivity | | Specificity | Sensitivity | | Specificity |
| Training cohort | 45.8% | (11/24) | 95.8% | 45.8% | (11/24) | 100% | 54.2% | (13/24) | 91.7% | 83.3% | (20/24) | 87.5% |
| | | | (23/24) | | | (24/24) | | | (22/24) | | | (21/24) |
| Tumor invasion | | | | | | | | | | | | |
| NMIBC | 21.4% | (3/14) | | 57.1% | (8/14) | | 64.3% | (9/14) | | 78.6% | (11/14) | |
| Grade of urothelial cancer | | | | | | | | | | | | |
| Low | 16.7% | (1/6) | | 50.0% | (3/6) | | 66.7% | (4/6) | | 66.7% | (4/6) | |
| High | 58.8% | (10/17) | | 47.1% | (8/17) | | 52.9% | (9/17) | | 94.1% | (16/17) | |
| Cytology | | | | | | | | | | | | |
| Negative^{a} | 43.8 | (7/16) | | 50.0% | (8/16) | | 56.3% | (9/16) | | 81.3% | (13/16) | |
| Positive | 50.0% | (4/8) | | 37.5% | (3/8) | | 50.0% | (4/8) | | 87.5% | (7/8) | |
| Independent cohort | 35.0% | (21/60) | 91.3% | 51.7% | (31/60) | 88.5% | 35.0% | (21/60) | 83.6% | 81.7% | (49/60) | 74.3% |
| | | | (167/183) | | | (162/183) | | | (153/183) | | | (136/183) |
| Tumor invasion | | | | | | | | | | | | |
| NMIBC | 44.7% | (21/47) | | 44.7% | (21/47) | | 40.4% | (19/47) | | 80.9% | (38/47) | |
| Grade of urothelial cancer | | | | | | | | | | | | |
| Low | 40.0% | (6/15) | | 33.3% | (5/15) | | 33.3% | (5/15) | | 80.0% | (12/15) | |
| High | 39.5% | (15/38) | | 50.0% | (19/38) | | 39.5% | (15/38) | | 78.9% | (30/38) | |
| Cytology | | | | | | | | | | | | |
| Negative^{a} | 41.2% | (7/17) | | 52.9% | (9/17) | | 23.5% | (4/17) | | 82.4% | (14/17) | |
| Positive | 35.0% | (14/40) | | 47.5% | (19/40) | | 40.0% | (16/40) | | 80.0% | (32/40) | |
| Combined cohort | 38.1% | (32/84) | 91.9% | 50.0% | (42/84) | 89.9% | 40.5% | (34/84) | 84.5% | 82.1% | (69/84) | 75.8% |
| | | | (190/207) | | | (186/207) | | | (175/207) | | | (157/207) |
| Tumor invasion | | | | | | | | | | | | |
| NMIBC | 39.3% | (24/61) | | 47.5% | (29/61) | | 45.9% | (28/61) | | 80.3% | (49/61) | |
| Grade of urothelial cancer | | | | | | | | | | | | |
| Low | 33.3% | (7/21) | | 38.1% | (8/21) | | 42.9% | (9/21) | | 76.2% | (16/21) | |
| High | 45.6% | (25/55) | | 49.1% | (27/55) | | 43.6% | (24/55) | | 83.6% | (46/55) | |
| Cytology | | | | | | | | | | | | |
| Negative^{a} | 42.4% | (14/33) | | 51.5% | (17/33) | | 39.4% | (13/33) | | 81.8% | (27/33) | |
| Positive | 37.5% | (18/48) | | 45.8% | (22/48) | | 41.7% | (20/48) | | 81.3% | (39/48) | |

Abbreviations: NMIBC, non-muscle invasive bladder cancer; MIBC, muscle invasive bladder cancer.
^{a}Negative cytology from our cohorts includes atypical urothelial cells and suspicious urothelial cancer cells.

TABLE 3
SEQUENCES OF PRIMERS FOR Q-RT-PCR
USED IN THE PRESENT STUDY

| GENE | FORWARD | REVERSE |
|---|---|---|
| NAME | PRIMER | PRIMER |
| CD24 | CTGGCACT | GAGTGAGA |
| | GCTCCTAC | CCACGAAG |
| | (SEQ ID | (SEQ ID |
| | NO: 1) | NO: 2) |
| SOX2 | CCCACCTA | TGGAGTGG |
| | CAGCATGT | GAGGAAGA |
| | CCTACTC | GGTAAC |
| | (SEQ ID | (SEQ ID |
| | NO: 3) | NO: 4) |
| ALDH1A1 | TGTTAGCT | TTCTTAGC |
| | GATGCCGA | CCGCTCAA |
| | CTTG | CACT |
| | (SEQ ID | (SEQ ID |
| | NO: 5) | NO: 6) |
| Bmi1 | CGTGTATT | TTCAGTGG |
| | GTTCGTTA | TCTGGTCT |
| | CCTGGA | TGT |
| | (SEQ ID | (SEQ ID |
| | NO: 7) | NO: 8) |
| OCT4 | GTCCGAGT | CTCAGTTT |
| | GTGGTTCT | GAATGCAT |
| | GTA | GGGA |
| | (SEQ ID | (SEQ ID |
| | NO: 9) | NO: 10) |
| LGR5 | GATGTTGC | TTTCCCGC |
| | TCAGGGTG | AAGACGTA |
| | GACT | ACTC |
| | (SEQ ID | (SEQ ID |
| | NO: 11) | NO: 12) |
| NANOG | CAGCTGTG | ACACCATT |
| | TGTACTCA | GCTATTCT |
| | ATGATAGA | TCGGCCAG |
| | TTT | TTG |
| | (SEQ ID | (SEQ ID |
| | NO: 13) | NO: 14) |
| Δ Np63 | ACCTGGAA | ACGAGGAG |
| | ACAATGCC | CCGTTCTG |
| | CAGA | AATC |
| | (SEQ ID | (SEQ ID |
| | NO: 15) | NO: 16) |
| CK14 | GGCCTGCT | GTCCACTG |
| | GACATCAA | TGGCTGTG |
| | AGAC | AGAA |
| | (SEQ ID | (SEQ ID |
| | NO: 17) | NO: 18) |
| CD133 | TGGGGCTG | TGCCACGG |
| | CTGTTTAT | GCCATAGA |
| | TATTCT | AGATG |
| | (SEQ ID | (SEQ ID |
| | NO: 19) | NO: 20) |
| CD44 | AGAAGGTG | AAATGCAC |
| | TGGGCAGA | CATTTCCT |
| | AGAA | GAGA |
| | (SEQ ID | (SEQ ID |
| | NO: 21) | NO: 22) |
| CD49f | CGAAACCA | CTTGGATC |
| | AGGTTCTG | TCCACTGA |
| | AGCCCA | GGCAGT |
| | (SEQ ID | (SEQ ID |
| | NO: 23) | NO: 24) |
| CD90 | CGCTCTCC | CAGGCTGA |
| | TGCTAACA | ACTCGTAC |
| | GTCTT | TGGA |
| | (SEQ ID | (SEQ ID |
| | NO: 25) | NO: 26) |
| HMGA2 | AAAGCAGC | TGTTGTGG |
| | TCAAAAGA | CCATTTCC |
| | AAGCA | TAGGT |
| | (SEQ ID | (SEQ ID |
| | NO: 27) | NO: 28) |
| YAP1 | ACCACAGC | TGGCTTGT |
| | TCAGC AT | TCCCATCC |
| | CTTCG | ATCAG |
| | (SEQ ID | (SEQ ID |
| | NO: 29) | NO: 30) |
| ABCG2 | AGCTGCAA | TCCAGACA |
| | GGAAAGAT | CACCACGG |
| | CCAA | ATAA |
| | (SEQ ID | (SEQ ID |
| | NO: 31) | NO: 32) |
| β-actin | TTCTACAA | GGGGTGTT |
| | TGAGCTGC | GAAGGTCT |
| | GTGTG | CAAA |
| | (SEQ ID | (SEQ ID |
| | NO: 33) | NO: 34) |

QUANTITATIVE RT-PCR WAS DONE AT 50° C. FOR 2 MIN, 95° C. FOR 10 MIN, FOLLOWED BY 40 CYCLES AT 95°C. FOR 15 SEC AND 58° C. FOR 1 MIN.

TABLE 6
SENSITIVITY AND SPECIFICITY OF 3 GENES PANEL IN COMBINED COHORT
(TRAINING AND VALIDATION, 84 CASES AND 207 CONTROLS)

| MARKERS | SENSITIVITY (%) | SPECIFICITY (%) | PLR | 95% CI | NLR | 95% CI |
|---|---|---|---|---|---|---|
| NANOG/CD24/CD49f | 82.1 | 75.8 | 3.40 | 2.71-4.08 | 0.24 | 0.15-0.36 |
| NANOG | 50.0 | 89.9 | 4.64 | 2.77-7.83 | 0.67 | 0.59-0.78 |
| CD24 | 38.1 | 91.8 | 2.62 | 1.74-3.90 | 0.70 | 0.59-0.83 |
| CD49f | 40.5 | 84.5 | 4.93 | 3.18-7.67 | 0.56 | 0.47-0.67 |

PPV, POSITIVE PREDICTIVE VALUE; PLR, POSITIVE LIKELIHOOD RATIO; NLR, NEGATIVE LIKELIHOOD RATIO; 95% CI, 95% CONFIDENCE INTERVAL.

TABLE 4
THE SENSITIVITY AND SPECIFICITY OF THE 15 GENES FOR CANCER DETECTION USING URINE (TRAINING COHORT)

| | EXPRESSION LEVEL (MEAN_ + SEM) | | | | | | |
|---|---|---|---|---|---|---|---|
| | CONTROL | CANCER | P-value | AUC | Cut-off | SENSITIVITY (%) | SPECIFICITY (%) |
| NANOG | 0.001 ± 0.001 | 0.202 ± 0.086 | 0.024 | 0.711 | 0.014 | 45.8 | 100.0 |
| CD24 | 0.006 ± 0.002 | 0.035 ± 0.012 | 0.020 | 0.627 | 0.022 | 45.8 | 95.8 |
| CD49 | 0.039 ± 0.011 | 0.250 ± 0.096 | 0.034 | 0.771 | 0.115 | 54.2 | 91.7 |
| LGR5 | 0.0001 ± 0.0001 | 0.0287 ± 0.013 | 0.041 | 0.548 | 1.435E−04 | 29.2 | 87.5 |
| Np63 | 0.0005 ± 0.0004 | 0.016 ± 0.006 | 0.030 | 0.694 | 6.360E−05 | 62.5 | 83.3 |
| SOX2 | 0.305 ± 0.238 | 1.132 ± 0.238 | 0.018 | 0.628 | 0.387 | 58.3 | 83.3 |
| Bmi1 | 0.152 ± 0.048 | 0.700 ± 0.196 | 0.009 | 0.818 | 0.118 | 87.5 | 66.7 |
| ALDH1A1 | 0.011 ± 0.004 | 0.232 ± 0.173 | NS (0.209) | 0.626 | 0.140 | 29.2 | 100.0 |
| HMGA2 | 0.013 ± 0.006 | 0.592 ± 0.388 | NS (0.143) | 0.524 | 0.104 | 20.8 | 100.0 |
| CD44 | 0.011 ± 0.006 | 0.032 ± 0.018 | NS (0.273) | 0.538 | 0.062 | 16.7 | 95.8 |
| YAP1 | 0.004 ± 0.032 | 0.066 ± 0.032 | NS (0.170) | 0.530 | 6.210E−04 | 37.5 | 87.5 |
| OCT4 | 0.007 ± 0.004 | 0.038 ± 0.019 | NS (0.125) | 0.520 | 0.022 | 29.2 | 87.5 |
| CD133 | 0.008 ± 0.035 | 0.126 ± 0.046 | NS (0.436) | 0.623 | 0.019 | 70.8 | 58.3 |
| CK14 | 0.0001 ± 0.0004 | 0.0004 ± 0.0004 | NS (0.379) | 0.554 | 1.418E−08 | 62.5 | 58.3 |
| CD90 | 0.176 ± 0.046 | 0.525 ± 0.353 | NS (0.332) | 0.509 | 0.053 | 70.8 | 50.0 |

AUC, AREA UNDER THE CURVE. RED MARK INDICATES MOLECULES WITH SIGNIFICANTLY DIFFERENT EXPRESSION BETWEEN UCB AND CONTROL SUBJECTS

TABLE 5
SENSITIVITY AND SPECIFICITY OF COMBINATION MARKERS FOR CANCER DETECTION
USING URINE (TRAINING COHORT)

| MARKERS | SENSITIVITY (%) | SPECIFICITY (%) | PPV (%) | NPV (%) | P-value |
|---|---|---|---|---|---|
| 6 MARKERS | | | | | |
| NANOG/CD24/CD49f/LGR5/Np63/SOX2 | 95.8 | 50.0 | 65.7 | 92.3 | 0.001 |
| 5 MARKERS | | | | | |
| NANOG/CD24/CD49f/LGR5/Np63 | 95.8 | 62.5 | 71.9 | 93.8 | <0.001 |
| NANOG/CD24/CD49f/LGR5/SOX2 | 91.7 | 62.5 | 71.0 | 88.2 | <0.001 |
| 4 MARKERS | | | | | |
| NANOG/CD24/CD49f/LGR5 | 83.3 | 80.0 | 76.9 | 85.7 | <0.001 |
| NANOG/CD24/CD49f/Np63 | 95.8 | 75.0 | 79.3 | 94.7 | <0.001 |
| NANOG/CD24/CD49f/SOX2 | 91.7 | 70.8 | 75.9 | 89.5 | <0.001 |
| CD24/CD49f/LGR5/SOX2 | 91.7 | 70.8 | 75.9 | 89.5 | <0.001 |
| NANOG/CD24/LGR5/SOX2 | 87.5 | 70.8 | 75.0 | 85.0 | <0.001 |
| NANOG/CD24/LGR5/Np63 | 87.5 | 66.7 | 72.4 | 84.2 | <0.001 |
| NANOG/CD24/LGR5/SOX2 | 91.7 | 62.5 | 71.0 | 88.2 | <0.001 |
| 3 MARKERS | | | | | |
| NANOG/CD24/CD49f | 83.3 | 87.5 | 87.0 | 84.0 | <0.001 |
| NANOG/CD24/LGR5 | 70.8 | 83.3 | 81.0 | 74.1 | <0.001 |
| NANOG/CD24/SOX2 | 87.5 | 79.2 | 80.8 | 86.4 | <0.001 |
| NANOG/CD24/Np63 | 87.5 | 79.2 | 80.8 | 86.4 | <0.001 |
| NANOG/CD49f/Np63 | 87.5 | 79.2 | 80.8 | 86.4 | <0.001 |
| CD24/CD49f/LGR5 | 83.3 | 75.0 | 76.9 | 81.8 | <0.001 |
| CD24/CD49f/SOX2 | 91.7 | 70.8 | 75.9 | 89.5 | <0.001 |

PPV, POSITIVE PREDICTIVE VALUE; NPV, NEGATIVE PREDICTIVE VALUE. RED MARK INDICATES A CANDIDATE COMBINATION PANEL WITH WELL BALANCED DIAGNOSTIC ACCURACY.

1. A method comprising the step of detecting CD24, CD49f and NANOG in a urine sample obtained from a patient suspected of having urothelial carcinoma.

2. The method of claim 1, wherein the detection step comprises polymerase chain reaction (PCR).

3. The method of claim 2, wherein the PCR utilizes at least one primer comprising SEQ ID NOS: 1, 2, 23, 24, 13 and 14.

4. The method of claim 2, wherein the PCR comprises quantitative real time PCR (RT-PCR).

5. The method of claim 4, wherein the RT-PCR parameters comprise 50° C. for 2 min., 95° C. for 10 min., followed by 40 cycles at 95° C. for 15 sec. and 58° C. for 1 min.

6. The method of claim 1, further comprising detecting at least one of ALDH1A1, Bmi1, OCT4, CK14, CD133, CD44, CD90, HMGA2, YAP1, and ABCG2.

7. The method of claim 6, wherein the detecting step comprises PCR and utilizes at least one primer comprising SEQ ID NOS:5-10, 17-32.

8. The method of claim 1, further comprising treating the patient with one or more of surgery, radiation therapy, chemotherapy and immunotherapy.

9. The method of claim 8, wherein surgery comprises one or more of transurethral resection, radical cystectomy, partial cystectomy, lymph node dissection and urinary diversion.

10. The method of claim 8, wherein chemotherapy comprises cisplatin-based chemotherapy or carboplatin-based chemotherapy.

11. The method of claim 8, wherein immunotherapy comprises a checkpoint inhibitor.

12. The method of claim 11, wherein the checkpoint inhibitor comprises one or more of ipilimumab, nivolumab, pembrolizumab, atezolizumab, avelumab and durvalumab.

13. A method for detecting urothelial carcinoma in a patient comprising the step of detecting higher expression of CD24, CD49f and NANOG in a urine sample obtained from the patient relative to a control.

14. The method of claim 13, wherein the detection step comprises polymerase chain reaction (PCR).

15. The method of claim 14, wherein the PCR utilizes at least one primer comprising SEQ ID NOS: 1, 2, 23, 24, 13 and 14.

16. The method of claim 14, wherein the PCR comprises quantitative real time PCR (RT-PCR).

17. The method of claim 16, wherein the RT-PCR parameters comprise 50° C. for 2 min., 95° C. for 10 min., followed by 40 cycles at 95° C. for 15 sec. and 58° C. for 1 min.

18. The method of claim 13, further comprising detecting at least one of ALDH1A1, Bmi1, OCT4, CK14, CD133, CD44, CD90, HMGA2, YAP1, and ABCG2.

19. The method of claim 18, wherein the detecting step comprises PCR and utilizes at least one primer comprising SEQ ID NOS:5-10, 17-32.

20. A method for treating urothelial carcinoma in a patient comprising the steps of:
(a) detecting higher expression of CD24, CD49f and NANOG in a biological sample obtained from the patient relative to a control; and
(b) treating the patient with one or more of surgery, radiation therapy, chemotherapy and immunotherapy.

21. The method of claim 20, wherein surgery comprises one or more of transurethral resection, radical cystectomy, partial cystectomy, lymph node dissection and urinary diversion.

22. The method of claim 20, wherein chemotherapy comprises cisplatin-based chemotherapy or carboplatin-based chemotherapy.

23. The method of claim 20, wherein immunotherapy comprises a checkpoint inhibitor.

24. The method of claim 23, wherein the checkpoint inhibitor comprises one or more of ipilimumab, nivolumab, pembrolizumab, atezolizumab, avelumab and durvalumab.

25. The method of claim 20, wherein the biological sample comprises urine, blood, plasma, serum, and saliva.

26. The method of claim 20, wherein the detection step comprises polymerase chain reaction (PCR).

27. The method of claim 20, wherein the PCR utilizes at least one primer comprising SEQ ID NOS: 1, 2, 23, 24, 13 and 14.

28. The method of claim 27, wherein the PCR comprises quantitative real time PCR (RT-PCR).

29. The method of claim 28, wherein the RT-PCR parameters comprise 50° C. for 2 min., 95° C. for 10 min., followed by 40 cycles at 95° C. for 15 sec. and 58° C. for 1 min.

30. The method of claim 20, further comprising detecting at least one of ALDH1A1, Bmi1, OCT4, CK14, CD133, CD44, CD90, HMGA2, YAP1, and ABCG2.

31. The method of claim 30, wherein the detecting step comprises PCR and utilizes at least one primer comprising SEQ ID NOS:5-10, 17-32.


SEQUENCE LISTING (34 sequences)

SEQ ID NO: 1 - DNA, 16 bp, Artificial Sequence
  Forward primer for CD24
ctggcactgctcctac

SEQ ID NO: 2 - DNA, 16 bp, Artificial Sequence
  Reverse primer for CD24
gagtgagaccacgaag

SEQ ID NO: 3 - DNA, 23 bp, Artificial Sequence
  Forward primer for SOX2
cccacctacagcatgtcctactc

SEQ ID NO: 4 - DNA, 22 bp, Artificial Sequence
  Reverse primer for SOX2
tggagtgggaggaagaggtaac

SEQ ID NO: 5 - DNA, 20 bp, Artificial Sequence
  Forward primer for ALDH1A1
tgttagctgatgccgacttg

SEQ ID NO: 6 - DNA, 20 bp, Artificial Sequence
  Reverse primer for ALDH1A1
ttcttagcccgctcaacact

SEQ ID NO: 7 - DNA, 22 bp, Artificial Sequence
  Forward primer for Bmi1
cgtgtattgttcgttacctgga

SEQ ID NO: 8 - DNA, 22 bp, Artificial Sequence
  Reverse primer for Bmi1
ttcagtagtggtctggtcttgt

SEQ ID NO: 9 - DNA, 19 bp, Artificial Sequence
  Forward primer for OCT4
gtccgagtgtggttctgta

SEQ ID NO: 10 - DNA, 20 bp, Artificial Sequence
  Reverse primer for OCT4
ctcagtttgaatgcatggga

SEQ ID NO: 11 - DNA, 20 bp, Artificial Sequence
  Forward primer for LGR5
gatgttgctcagggtggact

SEQ ID NO: 12 - DNA, 20 bp, Artificial Sequence
  Reverse primer for LGR5
tttcccgcaagacgtaactc

SEQ ID NO: 13 - DNA, 27 bp, Artificial Sequence
  Forward primer for NANOG
cagctgtgtgtactcaatgatagattt

SEQ ID NO: 14 - DNA, 27 bp, Artificial Sequence
  Reverse primer for NANOG
acaccattgctattcttcggccagttg

SEQ ID NO: 15 - DNA, 21 bp, Artificial Sequence
  Forward primer for Delta-Np63
acctggaaaacaatgcccaga

SEQ ID NO: 16 - DNA, 20 bp, Artificial Sequence
  Reverse primer for Delta-Np63
acgaggagccgttctgaatc

SEQ ID NO: 17 - DNA, 20 bp, Artificial Sequence
  Forward primer for CK14
ggcctgctgagatcaaagac

SEQ ID NO: 18 - DNA, 20 bp, Artificial Sequence
  Reverse primer for CK14
gtccactgtggctgtgagaa

SEQ ID NO: 19 - DNA, 22 bp, Artificial Sequence
  Forward primer for CD133
tggggctgctgtttattattct

SEQ ID NO: 20 - DNA, 22 bp, Artificial Sequence
  Reverse primer for CD133
tgccacaaaaccatagaagatg

SEQ ID NO: 21 - DNA, 20 bp, Artificial Sequence
  Forward primer for CD44
agaaggtgtgggcagaagaa

SEQ ID NO: 22 - DNA, 20 bp, Artificial Sequence
  Reverse primer for CD44
aaatgcaccatttcctgaga

SEQ ID NO: 23 - DNA, 22 bp, Artificial Sequence
  Forward primer for CD49f
cgaaaccaaggttctgagccca

SEQ ID NO: 24 - DNA, 22 bp, Artificial Sequence
  Reverse primer for CD49f
cttggatctccactgaggcagt

SEQ ID NO: 25 - DNA, 21 bp, Artificial Sequence
  Forward primer for CD90
cgctctcctgctaacagtctt

SEQ ID NO: 26 - DNA, 20 bp, Artificial Sequence
  Reverse primer for CD90
caggctgaactcgtactgga

SEQ ID NO: 27 - DNA, 21 bp, Artificial Sequence
  Forward primer for HMGA2
aaagcagctcaaaagaaagca

SEQ ID NO: 28 - DNA, 21 bp, Artificial Sequence
  Reverse primer for HMGA2
tgttgtggccatttcctaggt

SEQ ID NO: 29 - DNA, 21 bp, Artificial Sequence
  Forward primer for YAP1
acccacagctcagcatcttcg

SEQ ID NO: 30 - DNA, 21 bp, Artificial Sequence
  Reverse primer for YAP1
acccacagctcagcatcttcg

SEQ ID NO: 31 - DNA, 20 bp, Artificial Sequence
  Forward primer for ABCG2
agctgcaaggaaagatccaa

SEQ ID NO: 32 - DNA, 20 bp, Artificial Sequence
  Reverse primer for ABCG2
tccagacacaccacggataa

SEQ ID NO: 33 - DNA, 21 bp, Artificial Sequence
  Forward primer for Beta-actin
ttctacaatgagctgcgtgtg

SEQ ID NO: 34 - DNA, 20 bp, Artificial Sequence
  Reverse primer for Beta-actin
ggggtgttgaaggtctcaaa